(19) United States
(12) Patent Application Publication
(10) Pub. No.: US 2018/0214519 A1
(43) Pub. Date: Aug. 2, 2018

(54) COMBINATION THERAPIES FOR MODULATION OF HISTONE METHYL MODIFYING ENZYMES

(71) Applicant: Constellation Pharmaceuticals, Inc., Cambridge, MA (US)

(72) Inventors: Shilpi Arora, Bedford, MA (US); Srividya Balasubramanian, Lexington, MA (US); William D. Bradley, Winchester, MA (US); Patrick Trojer, Reading, MA (US)

(21) Appl. No.: 15/747,331

(22) PCT Filed: Jul. 24, 2015
(86) PCT No.: PCT/US2015/041877

§ 371 Date: Jan. 24, 2018

(51) Int. Cl. A61K 38/21; A61P 35/00; A61K 31/4545; A61K 31/4439

(52) U.S. Cl. CPC A61K 38/212; A61K 31/4439; A61K 31/4545; A61P 35/00

(57) ABSTRACT

Provided herein are pharmaceutical compositions comprising an EZH2 inhibitor and a type I interferon, processes for preparing such pharmaceutical compositions, and uses thereof in modulating the activity of histone methyl modifying enzymes.

### BACKGROUND

EZH2 (Enhancer of Zeste Homolog 2) is a histone lysine methyltransferase that has been implicated in the pathogenesis of both hematologic and non-hematologic malignancies. EZH2 catalyzes the transfer of one, two and three methyl-groups to lysine 27 of histone 3 (H3K27). EZH2 is the catalytic component of a large, multi-protein complex called polycomb repressive complex 2 (PRC2), which generally functions in transcriptional repression (Margueron, R., and Reinberg, D. (2011). The Polycomb complex PRC2 and its mark in life. Nature 469, 343-349.). Although in many instances transcriptional silencing by PRC2 is dependent on the catalytic activity of EZH2, it is clear that the physical association of the PRC2 complex with certain genes is also important in transcriptional suppression. The PRC2 complex can alternatively contain a closely related homolog of EZH2, known as EZH1. These two catalytic subunits of the PRC2 complex are the only enzymes known to catalyze H3K27 methylation. In addition to their catalytic activity, EZH1 and EZH2 are multi-domain proteins that mediate other biologic effects through protein-protein and protein-nucleic acid interactions. H3K27 di-methylation and tri-methylation (H3K27me2 and H3K27me3) correlate well with transcriptionally repressed genes, but H3K27 mono-methylation (H3K27me1) is found on transcriptionally active genes (Barski, A., et al. (2007). High-resolution profiling of histone methylations in the human genome. Cell 129, 823-837; Ferrari, K. J., et al. (2014). Polycomb-dependent H3K27me1 and H3K27me2 regulate active transcription and enhancer fidelity. Mol. Cell 53, 49-62.). Recent genetic studies suggest that EZH1-containing PRC2 controls H3K27me1 levels (Hidalgo, I., et al. (2012). Ezh1 is required for hematopoietic stem cell maintenance and prevents senescence-like cell cycle arrest. Cell Stem Cell 11, 649-662; Xie, H., et al. (2014). Polycomb repressive complex 2 regulates normal hematopoietic stem cell function in a developmental-stage-specific manner. Cell Stem Cell 14, 68-80.). This is consistent with a putative role of EZH1 in transcriptional elongation (Mousavi, K., et al. (2012). Polycomb protein Ezh1 promotes RNA polymerase II elongation. Mol. Cell 45, 255-262.). Thus, PRC2-dependent H3K27 methyltransferase activity is implicated in both transcriptional repression and activation, depending on the composition of the complex.

EZH2 (but not EZH1) is frequently overexpressed in human cancer. High levels of expression correlate with increased levels of H3K27me3, late stage disease and poor outcome, for instance in breast, lung, gastric, bladder, ovarian and prostate cancer, leukemia, lymphoma and multiple myeloma (Kleer, C. G., et al. (2003). EZH2 is a marker of aggressive breast cancer and promotes neoplastic transformation of breast epithelial cells. PNAS 100, 11606-11611; Varambally, S., et al. (2002). The polycomb group protein EZH2 is involved in progression of prostate cancer. Nature 419, 624-629; Weikert, S., et al. (2005). Expression levels of the EZH2 polycomb transcriptional repressor correlate with aggressiveness and invasive potential of bladder carcinomas. Int. J. Mol. Med. 16, 349-353.). A continuously increasing number of functional studies implicate PRC2 and specifically EZH2 in tumorigenesis, cancer progression and metastasis (Lu, C., et al. (2010). Regulation of tumor angiogenesis by EZH2. Cancer Cell 18, 185-197; Min, J., et al. (2010). An oncogene-tumor suppressor cascade drives metastatic prostate cancer by coordinately activating Ras and nuclear factor-kappaB. Nat. Med. 16, 286-294; Shi, J., et al. (2013). The Polycomb complex PRC2 supports aberrant self-renewal in a mouse model of MLL-AF9; Nras(G12D) acute myeloid leukemia. Oncogene 32, 930-938; Suvà, M.-L., et al. (2009). EZH2 is essential for glioblastoma cancer stem cell maintenance. Cancer Res. 69, 9211-9218; Wilson, B. G., et al. (2010). Epigenetic antagonism between polycomb and SWI/SNF complexes during oncogenic transformation. Cancer Cell 18, 316-328.). Recent genomic sequencing studies helped to elucidate the role of EZH2 in germinal center-derived lymphomas (Béguelin, W., et al. (2013). EZH2 is required for germinal center formation and somatic EZH2 mutations promote lymphoid transformation. Cancer Cell 23, 677-692.). As B-cells exit the germinal center their EZH2 levels decrease, promoting the expression of genes that ensure terminal differentiation. And conditional expression of an EZH2 mutant allele promoted lymphoid hyperplasia and lymphomagenesis by aberrantly repressing B-cell differentiation genes. The role of EZH2 in the development of germinal center-derived lymphomas has been further substantiated by the discovery of recurrent, monoallelic mutations in the gene encoding EZH2 in 15-25% of germinal center B-cell-like diffuse large B-cell lymphomas (GCB-DLBCL) and in 12-22% of follicular lymphomas (FL) (Ryan, et al. (2011). EZH2 codon 641 mutations are common in BCL2-rearranged germinal center B cell lymphomas. PLoS ONE 6, e28585; Morin, R. D., et al. (2011). Frequent mutation of histone-modifying genes in non-Hodgkin lymphoma. Nature 476, 298-303; Lohr, J. G., et al. (2012). Discovery and prioritization of somatic mutations in diffuse large B-cell lymphoma (DLBCL) by whole-exome sequencing. Proc. Natl. Acad. Sci. U.S.A. 109, 3879-3884; Guo, S., et al. (2014). EZH2 mutations in follicular lymphoma from different ethnic groups and associated gene expression alterations. Clin. Cancer Res. 20, 3078-3086; Bödör, C., et al. (2011). EZH2 Y641 mutations in follicular lymphoma. Leukemia 25, 726-729; Morin, R. D., et al. (2010). Somatic mutations altering EZH2 (Tyr641) in follicular and diffuse large B-cell lymphomas of germinal-center origin. Nat. Genet. 42, 181-185.). Recurrent EZH2 mutations have also been found with low frequency in melanoma (Hodis, E., et al. (2012). A Landscape of Driver Mutations in Melanoma. Cell 150, 251-263.). Recurrent mutations in EZH2 affect the amino acid residues Y641, A677 and A687 and alter the substrate specificity of the enzyme, making it more efficient in the conversion of H3K27 from a di-methylated to a tri-methylated state (Majer, C. R., et al. (2012). A687V EZH2 is a gain-of-function mutation found in lymphoma patients. FEBS Lett. 586, 3448-3451; McCabe, M. T., et al. (2012). Mutation of A677 in histone methyltransferase EZH2 in human B-cell lymphoma promotes hypertrimethylation of histone H3 on lysine 27 (H3K27). PNAS 109, 2989-2994; Sneeringer, C. J., et al. (2010). Coordinated activities of wild-type plus mutant EZH2 drive tumor-associated hypertrimethylation of lysine 27 on histone H3 (H3K27) in human B-cell lymphomas. Proc. Natl. Acad. Sci. U.S.A. 107, 20980-20985; Wigle, T. J., et al. (2011). The Y641C mutation of EZH2 alters substrate specificity for histone H3 lysine 27 methylation states. FEBS Lett. 585, 3011-3014; Yap, D. B., et al. (2011). Somatic mutations at EZH2 Y641 act dominantly through a mechanism of selectively altered PRC2 catalytic activity, to increase H3K27 trimethylation. Blood 117, 2451-2459.). Consequently, malignant cells containing one of these mutations exhibit higher global levels of H3K27me3 than those with the wild type enzyme. The dependence of these lymphomas on the heightened catalytic activity of the mutated enzyme is reflected in their sensitivity to highly selective inhibitors of EZH2 (Bradley, W. D., et al. (2014). EZH2 Inhibitor Efficacy in Non-Hodgkin's Lymphoma Does Not Require Suppression of H3K27 Monomethylation. Chemistry & Biology 21, 1463-1475; Diaz, E., et al. (2012). Development and validation of reagents and assays for EZH2 peptide and nucleosome high-throughput screens. J Biomol Screen 17, 1279-1292; Garapaty-Rao, S., et al. (2013). Identification of EZH2 and EZH1 Small Molecule Inhibitors with Selective Impact on Diffuse Large B Cell Lymphoma Cell Growth. Chemistry & Biology 20, 1329-1339; Knutson, S. K., et al. (2013). Durable tumor regression in genetically altered malignant rhabdoid tumors by inhibition of methyltransferase EZH2. Proc. Natl. Acad. Sci. U.S.A. 110, 7922-7927; Knutson, S. K., et al. (2012). A selective inhibitor of EZH2 blocks H3K27 methylation and kills mutant lymphoma cells. Nat Chem Biol 8, 890-896; Konze, K. D., et al. (2013). An Orally Bioavailable Chemical Probe of the Lysine Methyltransferases EZH2 and EZH1. ACS Chem. Biol. 8, 1324-1334; McCabe, M. T., et al. (2012). EZH2 inhibition as a therapeutic strategy for lymphoma with EZH2-activating mutations. Nature 492, 108-112; Nasveschuk, C. G., et al. (2014). Discovery and Optimization of Tetramethylpiperidinyl Benzamides as Inhibitors of EZH2. ACS Med Chem Lett 5, 378-383; Qi, W., et al. (2012). Selective inhibition of Ezh2 by a small molecule inhibitor blocks tumor cells proliferation. Proc. Natl. Acad. Sci. U.S.A. 109, 21360-21365.). While the anti-tumor activity of EZH2 inhibitors is most consistently observed in models of lymphoma with activating mutations in EZH2, there are models of lymphoma and other malignancies that are sensitive to EZH2 inhibition but that contain only wild type EZH2 (Bradley, W. D., et al. (2014). EZH2 Inhibitor Efficacy in Non-Hodgkin's Lymphoma Does Not Require Suppression of H3K27 Monomethylation. Chemistry & Biology 21, 1463-1475; McCabe, M. T., et al. (2012). EZH2 inhibition as a therapeutic strategy for lymphoma with EZH2-activating mutations. Nature 492, 108-112.).

EZH2 is regarded as an oncogene in certain cancer types. Efficacy in models of hematological malignancies and solid tumors has been shown with pharmacological inhibition of EZH2. See e.g., WO 2013/120104 and WO 2014/124418. Given its role in the regulation of diverse biological processes, and the therapeutic benefits associated with its inhibition, EZH2 remains an attractive target for modulation.

### SUMMARY

It has now been found that administration of an EZH2 inhibitor and a type I interferon synergistically treat cancer. See e.g., FIG. 5, which illustrates the sensitivity of lymphoma cells to treatment with both an EZH2 inhibitor and a type I interferon, FIG. 13 which illustrates a melanoma cell line sensitive to treatment with both an EZH2 inhibitor and a type I interferon, FIG. 14 which illustrates a multiple myeloma cell line sensitive to treatment with both an EZH2 inhibitor and a type I interferon, and FIG. 15 illustrates a lung adenocarcinoma cell line sensitive to treatment with both an EZH2 inhibitor and a type I interferon.

It has now also been found that certain interferon responses, such as in vitro cell growth inhibition, in vivo tumor growth inhibition in a mouse xenograft model, and full induction of interferon stimulated genes and their corresponding proteins, were only elicited upon treatment with the combination of an EZH2 inhibitor and a type I interferon, and not by each of these agents alone. See e.g., FIG. 6-8.

Based on these results, provided herein are methods of treating a subject with cancer by administering to the subject an effective amount of an EZH2 inhibitor and an effective amount of a type I interferon.

Also provided herein are pharmaceutical compositions comprising an EZH2 inhibitor and a type I interferon.

### BRIEF DESCRIPTION OF THE FIGURES

FIG. 1 illustrates induction of the interferon signaling pathway in KARPAS-422 cells upon addition of an EZH2 inhibitor, where FIG. 1a illustrates KARPAS-422 temporal sensitivity, FIG. 1b illustrates viable cells, FIG. 1c illustrates a heatmap representation, FIG. 1d represents a gene set enrichment analysis, and FIG. 1e illustrates a heatmap representation of differential expression of gene groups within the interferon signaling pathway in KARPAS-422 from an RNA-sequencing dataset.

FIG. 2 illustrates induction of interferon response genes in KARPAS-422 cells upon addition of an EZH2 inhibitor, where FIG. 2a illustrates KARPAS-422 cells treated with 0.2% DMSO or 1.5 or 20 μM EZH2 inhibitor, FIG. 2b illustrates an analysis via qPCR using a type I interferon gene specific qPCR array.

FIG. 3 illustrates gene expression changes upon treatment with an EZH2 inhibitor.

FIG. 4 illustrates molecular induction of interferon response in KARPAS-422 cells upon addition of an EZH2 inhibitor and an EZH2 inhibitor together with a type I interferon, where FIG. 4a illustrates KARPAS-422 cells treated with 0.15% DMSO, 1.5 or 15 μM EZH2 inhibitor, FIG. 4b shows KARPAS-422 cells treated with 0.1% BSA control, or 10 or 1000 U/ml interferon α2a, β1, or γ for 1 hour before harvesting, FIG. 4c and FIG. 4d show KARPAS-422 cells co-treated with a titrations of EZH2 inhibitor and interferon (IFN) α2a, and FIG. 4e shows Bliss independence volume score.

FIG. 5 illustrates the sensitivity of a panel of non-Hodgkin lymphoma cells to treatment with an EZH2 inhibitor, a type I interferon or the combination of both agents, where FIG. 5a illustrates cell models of non-Hodgkin's lymphoma (NHL) and FIG. 5b illustrates Bliss independence volume score.

FIG. 6 illustrates cell growth inhibition in RL lymphoma cells elicited only by the combination of both an EZH2 inhibitor and a type I interferon, where FIG. 6a illustrates RL cells treated with EZH2 inhibitor and then IFN α2a, FIG. 6b illustrates RL cells were treated with titrations of both EZH2 inhibitor and interferon α2a, and FIG. 6c illustrates the effect of a titration of either IFN α2a or IFN γ.

FIG. 7 illustrates the synergistic relationship between an EZH2 inhibitor and a type I interferon on a transcriptional and protein level in RL lymphoma cells, where FIG. 7a illustrates pretreatment of RL cells with EZH2 inhibitor before application of IFN and FIG. 7b illustrates a western blot analysis.

FIG. 8 illustrates the in vivo efficacy in RL xenografts of the combination of EZH2 inhibitor and a type I interferon, where FIG. 8a illustrates treatment of SCID mice, FIG. 8b illustrates the analysis of H3K27me3 in palpable tumors, and FIG. 8c illustrates fold induction.

FIG. 9 illustrates dose dependent induction of lymphoma cell killing through EZH2 inhibitor combination with interferons and suppression of this phenotype by the addition of the JAK kinase inhibitor ruxolitinib, where FIG. 9a illustrates RL cells co-treated with EZH2 inhibitor and IFN α2a, FIG. 9b illustrates the addition of 1 μM ruxolitinib, and FIG. 9c represents an aliquot of cells were also processed for cell cycle analysis.

FIG. 10 illustrates that the viability defect is caused by the induction of apoptosis, where FIG. 10a and FIG. 10b illustrate cells processed via Annexin V and propidium iodide staining, then quantitated with a Guava cell analyzer.

FIG. 11 illustrates the impact on cell cycle progression, where FIG. 11a and FIG. 11b display data for full titration.

FIG. 12 illustrates the synergistic relationship between an EZH2 inhibitor and a type I interferon on a transcriptional and protein level in RL lymphoma cells and that the JAK kinase inhibitor ruxolitinib can suppress this synergistic interferon response, where FIG. 12a illustrates RL cells treated with EZH2 inhibitor, FIG. 12b is a representation of IFI27 and IFI6 genes from type I interferon qPCR array, and FIG. 12c illustrates RNA samples generated in protein lysates.

FIG. 13 illustrates a melanoma cell line sensitive to treatment with an EZH2 inhibitor, resulting in the transcriptional activation of interferon related genes, where FIG. 13a illustrates Colo-829 melanoma cells treated with EZH2 inhibitor, and FIG. 13b and FIG. 13c illustrate RNA extraction and sequencing.

FIG. 14 illustrates a multiple myeloma cell line sensitive to treatment with an EZH2 inhibitor, resulting in the transcriptional activation of interferon related genes, where FIG. 14a shows RPMI-8226 multiple myeloma cells treated with EZH2 inhibitor, FIG. 14b illustrates RPMI-8226 cells monitored for H3K27me3 and total H3 levels by MSD ELISA, and FIG. 14c and FIG. 14d illustrate RNA extraction and sequencing.

FIG. 15 illustrates a lung adenocarcinoma cell line sensitive to treatment with both an EZH2 inhibitor and an inhibitor of the epidermal growth factor receptor (EGFR) tyrosine kinase, where FIG. 15a is an experimental design schematic, FIG. 15b illustrates lung adenocarcinoma PC9 NucRed cells pre-treated with EZH2 inhibitor and then erlotinib, FIG. 15c is an experimental design schematic, FIG. 15d illustrates treatment with DMSO, and FIG. 15e illustrates a reduction in cell number in PC9 cells.

FIG. 16 illustrates transcriptomic response of a lung adenocarcinoma cell line with both an EZH2 inhibitor and an EGFR inhibitor (erlotinib)in comparison to erlotinib alone, where FIG. 16a illustrates PC9 cells pre-treated with EZH2 inhibitor, FIG. 16b illustrates alteration of the EGFR signaling pathway, FIG. 16c illustrates altered interferon-related pathways, FIG. 16d illustrates a gene set enrichment analysis plot for EGFR pathway, and FIG. 16e illustrates a gene set enrichment analysis plot for IFN pathway.

FIG. 17 illustrates molecular phenotype response with an EZH2 inhibitor, a type I interferon, and an EGFR inhibitor (erlotinib) in comparison to EZH2 inhibitor and erlotinib only, where FIG. 17a and FIG. 17b illustrate PC9 NucRed cells pre-treated with EZH2 inhibitor and erlotinib only or erlotinib only and IFN α2a.

FIG. 18 expands on the data in FIG. 17, where FIG. 18a represents a combination of bottom graphs from FIG. 16a and FIG. 16b, and FIG. 18b illustrates a reduction of DTP number and erlotinib-resistant cell outgrowth when compared to EZH2 inhibitor treated cells alone.

### DETAILED DESCRIPTION

In one aspect, present disclosure provides a method of treating cancer in a subject in need thereof, comprising the step of administering to the subject in need thereof an effective amount of an EZH2 inhibitor and an effective amount of a type I interferon.

It will be understood that unless otherwise indicated, the administrations described herein include administering a described EZH2 inhibitor prior to, concurrently with, or after administration of a type I interferon described herein. Thus, simultaneous administration is not necessary for therapeutic purposes. In one aspect, however, the EZH2 inhibitor is administered concurrently with the type I interferon.

The type I interferons described herein include e.g., the alpha and beta interferons encoded by genes selected from IFNA1, IFNA2, IFNA4, IFNA5, IFNA6, IFNA7, IFNA8, IFNA10, IFNA13, IFNA14, IFNA16, IFNA17, IFNA21, IFNB1, IFNW1, IFNE, and IFNK. Thus, in one aspect, the type I interferon in the methods described herein is an alpha or beta interferon encoded by genes selected from IFNA1, IFNA2, IFNA4, IFNA5, IFNA6, IFNA7, IFNA8, IFNA10, IFNA13, IFNA14, IFNA16, IFNA17, IFNA21, IFNB1, IFNW1, IFNE, and IFNK. In one alternative aspect, the type I interferon is interferon (IFN)-alpha-2a, interferon-alpha-2b, or interferon-beta-1a. In another alterantive aspect, the type I interferon is pegylated, such as e.g., pegylated interferon-alpha-2a, pegylated interferon-alpha-2b (Peg-Intron), and pegylated interferon-beta-1a. In another alternative aspect, the type I interferon is peginterferon alfa-2a (Pegasys) or peginterferon alfa-2b (Peg-Intron).

EZH2 inhibitors described herein include e.g., small molecules or biologics that are capable of inhibiting EZH2 methyltransferase activity. Inhibition can be measured in vitro, in vivo, or from a combination thereof. In one aspect, the EZH2 inhibitors in the methods described herein are selected from EPZ-6438, EPZ005687, EPZ011989, EI1, GSK126, GSK343, and UNC1999, as well as from those described in WO 2013/075083, WO 2013/075084, WO 2013/078320, WO 2013/120104, WO 2014/124418, WO 2014/151142, and WO 2015/023915. In one alternative aspect, the EZH2 inhibitors in the methods described herein are selected from

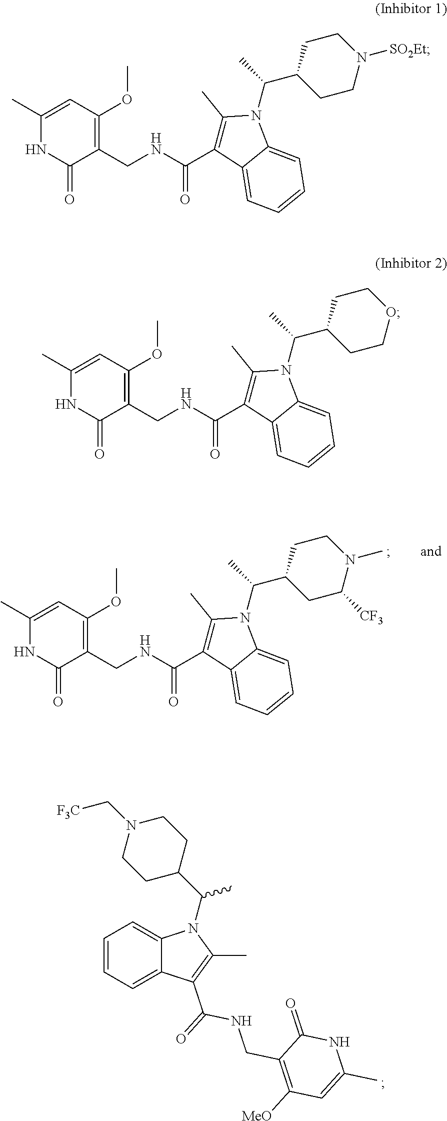

or a pharmaceutically acceptable salt thereof. In another alternative aspect, the EZH2 inhibitors in the methods described herein are

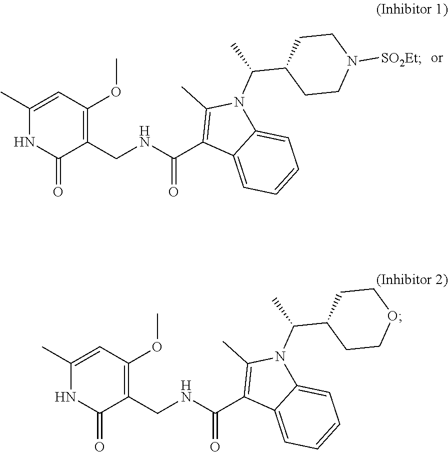

or a pharmaceutically acceptable salt thereof. In another alternative aspect, the EZH2 inhibitors in the methods described herein are

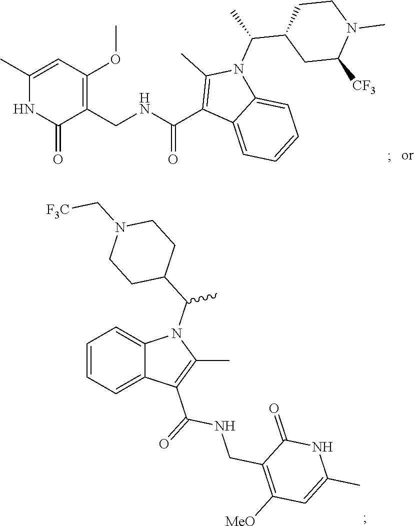

or a pharmaceutically acceptable salt thereof.

As described herein, the amount of an EZH2 inhibitor and a type I interferon is such that together, they elicit a synergistic effect to measurably modulate a histone methyl modifying enzyme, inhibit EZH2 and/or treat one or more cancers as described herein in a biological sample or in a patient.

As used herein, the terms “treatment,” “treat,” and “treating” refer to reversing, alleviating, or inhibiting the progress of a cancer, or one or more symptoms thereof, as described herein. Exemplary types of cancer include e.g., adrenal cancer, acinic cell carcinoma, acoustic neuroma, acral lentiginous melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogenous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, primary peritoneal cancer, prostate cancer, pancreatic cancer, pharyngeal cancer, pseudomyxoma peritonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor.

In one aspect, the cancer treated by the combination of an EZH2 inhibitor and a type I interferon is selected from melanoma, prostate cancer, breast cancer, colon cancer, ovarian cancer, bladder cancer, lung adenocarcinoma, and carcinoma of the pancreas. In another aspect, the cancer is selected from multiple myeloma, Hodgkin's lymphoma, non-Hodgkin's lymphoma, chronic lymphocytic leukemia, adult acute myeloid leukemia (AML), acute B lymphoblastic leukemia (B-ALL), and T-lineage acute lymphoblastic leukemia (T-ALL). In another aspect, the cancer treated is selected from Hodgkin's lymphoma, non-Hodgkin's lymphoma, chronic lymphocytic leukemia, and multiple myeloma. In another aspect, the cancer treated is non-Hodgkin's lymphoma.

Other aspects of the present disclosure also related to a method of eliciting an interferon response in a subject in need thereof, comprising the step of administering to the subject an effective amount of an EZH2 inhibitor and an effective amount of a type I interferon, wherein the interferon response is not elicited by either the EZH2 inhibitor or the type I interferon alone. In one aspect, the interferon response is selected from tumor growth inhibition, cell growth inhibition /defect in cell cycle progression/ apoptosis, and/or induction of interferon stimulated genes and their corresponding proteins.

Pharmaceutical compositions comprising an EZH2 inhibitor and a type I interferon as described herein are also included.

Also included are the use of an EZH2 inhibitor and a type I interferon as described herein in the manufacture of a medicament for the treatment of one or more cancers described herein. Also included herein are pharmaceutical compositions comprising an EZH2 inhibitor and a type I interferon as described herein optionally together with a pharmaceutically acceptable carrier, in the manufacture of a medicament for the treatment of one or more cancers described herein. Also included is an EZH2 inhibitor for use in combination with a type I interferon for the treatment of a subject with cancer. Further included are pharmaceutical compositions comprising an EZH2 inhibitor and a type I interferon described herein, optionally together with a pharmaceutically acceptable carrier, for use in the treatment of one or more cancers described herein. Futher included are pharmaceutical compositions comprising an EZH2 inhibitor and a type I interferon as described herein optionally together with a pharmaceutically acceptable carrier for use in the treatment of one or more cancers described herein.

The term “pharmaceutically acceptable carrier, adjuvant, or vehicle” refers to a non-toxic carrier, adjuvant, or vehicle that does not adversely affect the pharmacological activity of the compound with which it is formulated, and which is also safe for human use. Pharmaceutically acceptable carriers, adjuvants or vehicles that may be used in the compositions of this disclosure include, but are not limited to, ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., microcrystalline cellulose, hydroxypropyl methylcellulose, lactose monohydrate, sodium lauryl sulfate, and crosscarmellose sodium), polyethylene glycol, sodium carboxymethylcellulose, polyacrylates, waxes, polyethylene-polyoxypropylene-block polymers, polyethylene glycol and wool fat.

Compositions and method of administration herein may be orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Other forms of administration are as described in WO 2013/075083, WO 2013/075084, WO 2013/078320, WO 2013/120104, WO 2014/124418, WO 2014/151142, and WO 2015/023915, the contents of which are incorporated herein by reference.

### EXEMPLIFICATION

While have described a number of embodiments of this, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

### General Methods

### Cell Culture

Cell lines were obtained from ATCC or DSMZ, and maintained in culture as per each vendor's recommended conditions. Each cell line was authenticated by STR analysis. Optimal seeding density for each cell line for growth in a 96 well plate was determined by seeding cells at various densities, then measuring viability using the Cell Titer Glo (CTG) assay (Promega) at days 0, 2, and 4. Doubling time for each cell density was determined, and the density with the shortest doubling time that maintained cells in exponential growth was used for subsequent assays.

### Compounds

The EZH2 inhibitors utilized in these studies were synthesized as previously described (Bradley, W. D., et al. (2014). EZH2 Inhibitor Efficacy in Non-Hodgkin's Lymphoma Does Not Require Suppression of H3K27 Monomethylation. Chemistry & Biology 21, 1463-1475.). Ruxolitinib and erlotinib were purchased from Selleck Chemicals. IFNα2a was purchased from ProSpec, and IFNβ1 and IFNγ from Millipore.

### Cell Viability Assays

For suspension cell lines, cells were seeded at their pre-determined optimal seeding density in a 96 well dish in a volume of 70-90 μl, depending on the number of compounds under investigation in a single assay (i.e. single agent assay versus combination assay). A 10× stock solution of the highest desired concentration of the compound of interest was made in growth media. In parallel, a solution containing the same % of diluent as the 10× compound concentration was made for each compound under investigation, also in growth media. Compound was diluted serially in the diluent/growth media solution, and 10 μl of 10× drug was added to the relevant wells of the 96 well dish containing cells. Diluent for EZH2 inhibitors and ruxolitinib was DMSO (Sigma), whereas diluent for IFN was 0.1% (w/v) bovine serum albumin (BSA, Sigma) in phosphate-buffered saline (PBS, Sigma). Cells were cultured in the presence of compound for 4 days, at which point 100 μl growth media was added, cells were triturated, and the cell density of the diluent only control treatment was determined. A split ratio was determined based on this density, such that the new cell density of diluent treated cells after splitting would match the original cell density from day 0. The same volume of cells for all wells was transferred to a new 96 well dish, the volume increased to 70-90 μl with fresh growth media, and 10 μl 10× compound added as described above. 50 μl CTG reagent was added to all wells containing cells that remained in the original 96 well dish, followed by a 30 min incubation on an orbital shaker, before a luminescence reading was obtained on a Perkin-Elmer Envision. To determine relative cell viability, the mean relative light unit (RLU) reading for all diluent-treated controls was determined, and RLU readings for all wells were divided by this mean value to generate a % viable cell metric. A mean % viable value was generated for each compound concentration tested, plotted versus concentration, and fit to a 4 parameter sigmoidal curve using Prism 6.0 (GraphPad). GI₅₀ (50% growth inhibition) values were determined via extrapolation from the sigmoidal curve fit, and represent the concentration at which cell viability is 50% of the diluent control value at a given time point. Each assay was performed at least 3 times, with % viable graphed±standard error of the mean (SEM).

For adherent cell lines, cells were seeded at their pre-determined optimal seeding density as described above. 24 hours later, compound was added to cells similar to above, and compound incubation proceeded for 4 days. After 4 days, media was aspirated from all wells, and cells were washed with 100 μl PBS. The PBS was then aspirated, and 50 μl Tryp-LE (Life Technologies) was added to each well, and incubated at 37° C. for at least 5 min, until cells in all wells detached from the plate surface. 150 μl growth media was added, cells were triturated, and further processed similar to suspension cells, as described above.

### Cell Cycle Analysis

In some instances, cell cycle analysis was performed in parallel to cell viability assays. After splitting forward a desired volume of cells, and before addition of CTG reagent for cell viability assays, a portion of cells from all wells was moved to new v-bottom 96 well plates (Corning). Plates were centrifuged at 1000×g for 5 min, media was removed, cells were resuspended gently in 150 μl ice cold PBS, centrifuged again, and PBS was removed. Ice cold 70% ethanol was added slowly drop wise to each well, and cells were resuspended gently. Plates were stored at 4° C. for at least 24 hours before proceeding. Plates were centrifuged, and ethanol was removed. Cells were gently resuspended in 150 μl PBS, centrifuged again, and PBS was removed. Finally, cells were resuspended in 150 μl staining solution (0.1% (v/v) Triton X-100, 20 μg/mL propidium iodide, 20 μg/mL RNase A in PBS), incubated for 30 minutes protected from light, then gently mixed one final time before acquisition of 2500 events on a Guava EasyCyte System using the Guava Express Pro software. Data was analyzed using standard protocols.

### Annexin V Staining

In some instances, Annexin V staining was also performed in parallel to cell viability assays. After splitting forward desired volume of cells, and before addition of CTG reagent for cell viability assays, a portion of cells from all wells were moved to new v-bottom 96 well plates (Corning). Plates were centrifuged at 1000×g for 5 min, media was removed, cells were resuspended gently in 150 μl ice cold PBS, centrifuged again, and PBS was removed. Cells were gently resuspended in 25 μl stain buffer (Trevingen, TACS Annexin V-FITC Kit), and incubated for 15 min at room temperature protected from light. Then cells were mixed with 125 μl binding buffer, and 2500 events acquired on a Guava EasyCyte System using the Guava Express Pro software. Data was analyzed using standard protocols.

### Bliss Independence Volume Analysis

When two compounds were tested in combination in cell viability assays as described above, the Bliss independence volume method was utilized to determine if the two compounds interacted synergistically, antagonistically, or additively (ibid). For combination assays, the same concentration of compound 1 was added to all wells in the same column, and the same concentration of compound 2 was added to all wells in the same row, with each drug titrated serially in appropriate diluent, such that each well on the plate received a unique combination of concentrations of both compounds with at least one well containing both diluents only. The fraction of cells affected (FA) for each condition was determined by normalizing the RLU values to the diluent/diluent control, and subtracting from 1. The predicted additive effect for each unique combination was determined using the Bliss independence formula applied to the single agent activity of each compound at that concentration: (FA_{compound 1}+FA_{compound 2})−(FA_{compound 1}* FA_{compound 2}). The Bliss score for each individual drug combination was determined by subtracting the predicted additive fraction affected from the experimentally determined fraction affected. Positive values indicate a synergistic response, negative values indicate an antagonistic response, and a null value indicates an additive response. The individual synergy and antagonism values at the 95% confidence interval were summed for each cell line. In relation to the % viable metric, % viable=1−FA.

### MSD ELISA

H3K27me3 and total H3 levels were determined via MSD ELISA as previously described (ibid).

### Transcriptomic Analysis

### Sample Generation

Cell lines were treated with compound or diluent as indicated for each experiment. At the end of the incubation period, cells were collected, pelleted via centrifugation at 500×g for 5 minutes, followed by aspiration of cell culture media, and direct lysis in either Trizol (Life Technologies) or buffer RLT (Qiagen). Lysates were snap frozen on dry ice, and stored at −80° C. until further processing. Trizol treated cell lysates were further purified using the manufacturer's protocol. For buffer RLT-treated cell lysates, RNA was purified using an RNeasy column kit (Qiagen) following the manufacturer's protocol, including the optional DNase treatment step. Following both methods, RNA concentration was determined via NanoDrop (Thermo).

### RNA-Sequencing

For RNA sequencing (RNA-seq) experiments, RNA was submitted to Ocean Ridge Biosciences (Palm Beach Gardens, Fla.) for quality control, library preparation and sequencing. Samples were processed as per the vendor's protocols (http://www.oceanridgebio.com/rna-sequencing.html). Data was processed as previously described (ibid).

### Gene Set Enrichment Analysis

Gene set enrichment analysis (GSEA) was performed using the GSEA software package (Subramanian, A., et al. (2005). Gene set enrichment analysis: A knowledge-based approach for interpreting genome-wide expression profiles. PNAS 102, 15545-15550; Mootha, V. K., et al. (2003). PGC-1α-responsive genes involved in oxidative phosphorylation are coordinately downregulated in human diabetes. Nat Genet 34, 267-273.), v2.0.12 (http://www.broadinstitute.org/gsea/index.jsp). Experimental gene lists pre-ranked by differential expression were compared against gene sets in the Molecular Signature Database (MSigDB), v4.0 (http://www.broadinstitute.org/gsea/msigdb/index.jsp). GSEA analyses were run using the weighted scoring option, meandiv normalization, and excluding MSigDB gene sets with more than 1500 or fewer than 15 member genes.

### Generation of Interferon Gene List

The list of interferon-associated genes used for heatmaps was generated by merging all known interferon, interferon receptor, JAK family kinase, and STAT coding genes, and for interferon-stimulated genes (ISGs), taking the union of the following interferon-associated gene sets from msigdb v4: BOSCO_INTERFERON_INDUCED_ANTIVIRAL_MODULE, BROWNE_INTERFERON_RESPONSIVE_GENES, REACTOME_INTERFERON_ALPHA_BETA_SIGNALING, DER_IFN_ALPHA_RESPONSE_UP, RADAEVA_RESPONSE_TO_IFNA1_UP, MOSERLE_IFNA_RESPONSE, and HECKER_IFNB1_TARGETS. Only genes with a log fold change of 1 relative to diluent, with a p-value of 0.05 or less are displayed.

### qPCR

For quantitative polymerase chain reaction (qPCR) experiments, 200-1000 ng RNA was converted to cDNA using SuperScript III Reverse Transcriptase and 250 ng random primers (Life Technologies) in a 20 μl reaction using the manufacturer's protocol. Following first strand synthesis, the concentration of cDNA was diluted to 10 ng/μl (assuming 100% conversion) in nuclease-free water (Qiagen). For manual qPCR, 2 μl cDNA was mixed with primers and probes targeting the indicated genes, and FastStart Universal Probe Master Mix (Roche) in a 10 μl reaction. qPCR reactions were run in triplicate or quadruplicate on a LightCycler (Roche) using standard hydrolysis probe protocols. Cₜ, ΔCₜ,and ΔΔCₜ values were determined automatically using standard methods. In some instances, a Type I IFN Response PCR Array was utilized (SAB Biosciences, catalog #PAHS-016ZA). For these arrays, cDNA was prepared as above, and qPCR reactions performed as per the manufacturer's protocol on a Stratagene MX3005p. To generate fold change values, the geometric mean of 5 different genes of reference were determined, and subsequent calculations performed as stated above. In all cases, data is represented ±SEM, with n=3 or 4 for qPCR, and n=2 for qPCR arrays.

All primers and FAM-labeled probes were purchased from IDT, UPL probes from Roche Universal Probe Library, and TaqMan primer/probesets from Life Technologies.

Primers and probes used in these studies:
MX1:
Forward = CGCCTTGGACCGCAGTTG,
Reverse = CTTGGAATGGTGGCTGGATG,
FAM-labeled probe = AGACTCCCACTCCCTGAAATCTGGA;
IFITM1:
Forward = CCGTGAAGTCTAGGGACAGG,
Reverse = AGGCTATGGGCGGCTACTA,
FAM-labeled probe = TGGGCATCCTCATGACCATTGGAT;
IFIT1:
Forward = CCATGAGTACAAATGGTGATGA,
Reverse = CATTCTGGCCTTTCAGGTGT,
FAM-labeled probe = TCCATTGATGACGATGAAATGCCTGA;
IFIT3:
Forward = GGGCAGTCATGAGTGAGGTC,
Reverse = AGGGCTGCCTCGTTGTTAC,
UPL probe #80;
IFI27:
Forward = CTCTCCTTCTTTGGGTCTGG,
Reverse = ACAGCCACAACTCCTCCAAT,
UPL probe #21
IFI6:
Forward = CTCCTCCAAGGTCTAGTGACG,
Reverse = CGACTGCGAGTCCTCCTC,
UPL probe #62; TNFSF10: TaqMan probe Hs00921974_m1
(Life Technologies, catalog #4331182);
ACTB: TaqMan probe Hs99999903_m1 (Life
Technologies, catalog #4448484);
TBP: TaqMan probe
Hs00427621_m1 (Life Technologies, catalog #4453320).

### Immunoblotting

Cells were treated as indicated, then harvested at desired time point by centrifugation at 500×g for 5 minutes, followed by aspiration of cell culture media, resuspension in ice cold PBS, and another centrifugation and aspiration. Cell pellets were resuspended in ice cold RIPA-500 buffer (50 mM Tris pH 7.4, 500 mM NaCl, 1% (v/v) Triton X-100, 0.5% (v/v) sodium deoxycholate, 0.1% (v/v) SDS), supplemented with protease (Roche, Complete mini) and phosphatase (Roche, PhosStop) inhibitors, and 1,000 U/ml benzonase (EMD), incubated on ice until there was no remaining viscosity (˜30 minutes), then centrifuged for 10 min at 20,000×g at 4° C. Protein concentration of supernatants were determined via BCA assay (Pierce), and concentrations normalized in lysis buffer before addition of Laemmli sample buffer (LSB), and heating to 70° C. for 10 min. Samples were resolved on 4-12% Bolt Bis-Tris gels using MES buffer (Life Technologies), then transferred to 0.2 μm nitrocellulose (Bio-Rad) using a wet transfer method (Bio-Rad) at 100 V for 1 h in Towbin buffer. Blots were blocked in 5% (w/v) non-fat dry milk or 5% (w/v) cold water fish gelatin (Sigma) dissolved in PBS for 30-60 min, then incubated overnight at 4° C. with antibodies directed against the indicated protein of interest diluted in 1% (w/v) gelatin in PBS plus 0.1% (v/v) Tween-20 (PBST). Blots were washed 3×5 min in PBST, then incubated with secondary antibodies conjugated with DyLight 800 (LiCor) or Alexa 680 (Jackson Immunoresearch), diluted in 1% (w/v) gelatin in PBST for 1 h at room temperature. Blots were washed again, as above, with a final 5 min wash in PBS before signal acquisition using the Odyssey imager (LiCor). Antibodies raised against the following proteins were purchased from Cell Signaling Technology: pSTAT1 (#9167), STAT1 (#9176), pSTAT2 (#4441), STAT2 (#4594), pSTAT3 (#4113), STAT3 (#4904), IFIT1 (#12082), IFITM1 (#13126), ISG15 (#2743), H3K27me3 (#9733), H3 (#3638), ACTB (#3700), and the antibody raised against IFIT3 was purchased from Abcam (ab76818).

### Xenograft Studies and Tumor Processing

Female CB17 SCID mice were inoculated subcutaneously in the right flank with RL tumor cells (1×10⁷) in 0.2 ml of PBS mixed 1:1 with Matrigel (BD) for tumor development. Treatments were started 7 d post-inoculation when average tumor size reached approximately 100 mm³. Each group consisted of 9 randomly assigned tumor-bearing mice. The mice were dosed with vehicle (10% DMSO+60% polyethylene glycol 400+30% ddH₂O), CPI-169 (200 mg/kg, sc, bid) Pegasys (Pegylated-interferon α2a, Roche, 1×10⁶ U/mouse, qw), or the combination of both treatments as per IACUC guidelines. 6 h following the application of the last dose, tumor samples were collected and analyzed by ELISA for H3K27me3 levels (see above). RNA was extracted from tumors to measure changes in gene expression by qPCR (see above).

### Drug Tolerant Persister Cell Generation

The non-small cell lung cancer (NSCLC) cell line PC9 was transduced with virus expressing nuclear-restricted RFP (NucRed, Essen Biosciences NucLight), and selected with zeocin (Invivogen). Drug tolerant persister (DTP) cells were generated similar to published methods (Sharma, S. V., et al. (2010). A chromatin-mediated reversible drug-tolerant state in cancer cell subpopulations. Cell 141, 69-80.). In brief, 5×10⁴ cells were seeded on 12 well tissue culture plates (Corning) for 48 hours, before application of a GI90 dose (1 μM) of the EGFR inhibitor erlotinib (Selleck Chemicals). Every 3-4 days, media was removed, cells were washed with PBS, and fresh growth media containing 1 μM erlotinib was added. DTPs are defined as cells maintaining red fluorescence, while remaining in a non-proliferative state. After a period of time, DTPs regain proliferative capacity, at which point they are deemed drug tolerant expanded persisters (DTEPs) (ibid). Cells were maintained in 12 well dishes in an Incucyte ZOOM (Essen Biosciences) positioned in a humidified tissue culture incubator for the length of each experiment. Whole well phase and fluorescent images were obtained every 12 hours by the Incucyte ZOOM, and cell number quantified using an automated algorithm defined from a training set of images that identifies red fluorescent signal (positive signal pseudocolored blue in figures). In each experiment, relative cell number is quantitated by dividing cell number at each time point by the initial cell number for each well. In some instances, PC9 NucRed cells were pretreated with EZH2 inhibitors, before counting and reseeding at 5×10⁴ cells per well for DTP generation assays as described above.

For transcriptomic analysis of PC9 cells, cells were pretreated for 8 days with 2.5 μM CPI-360, with one splitting event and reapplication of fresh drug at day 4. On day 8, without splitting, 0.1% (v/v) DMSO or 1 μM erlotinib was added directly to each vessel, mixed, and incubated for 6 or 24 hours before harvesting. Cells were harvested by aspiration of media, and direct application of buffer RLT to the cell culture plate. RNA was purified as described above.

### EXPERIMENTAL SECTION

### EZH2 Inhibitors Induce Interferon Response

It has now been found that treatment with an EZH2 inhibitor initiates interferon response signaling pathways.

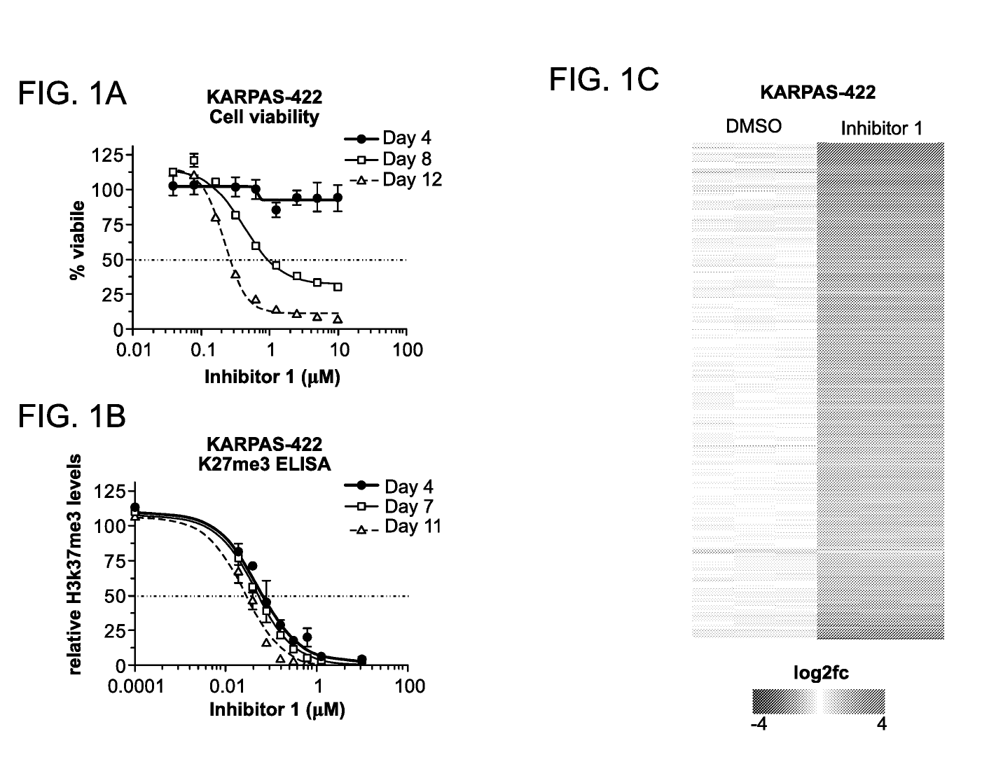

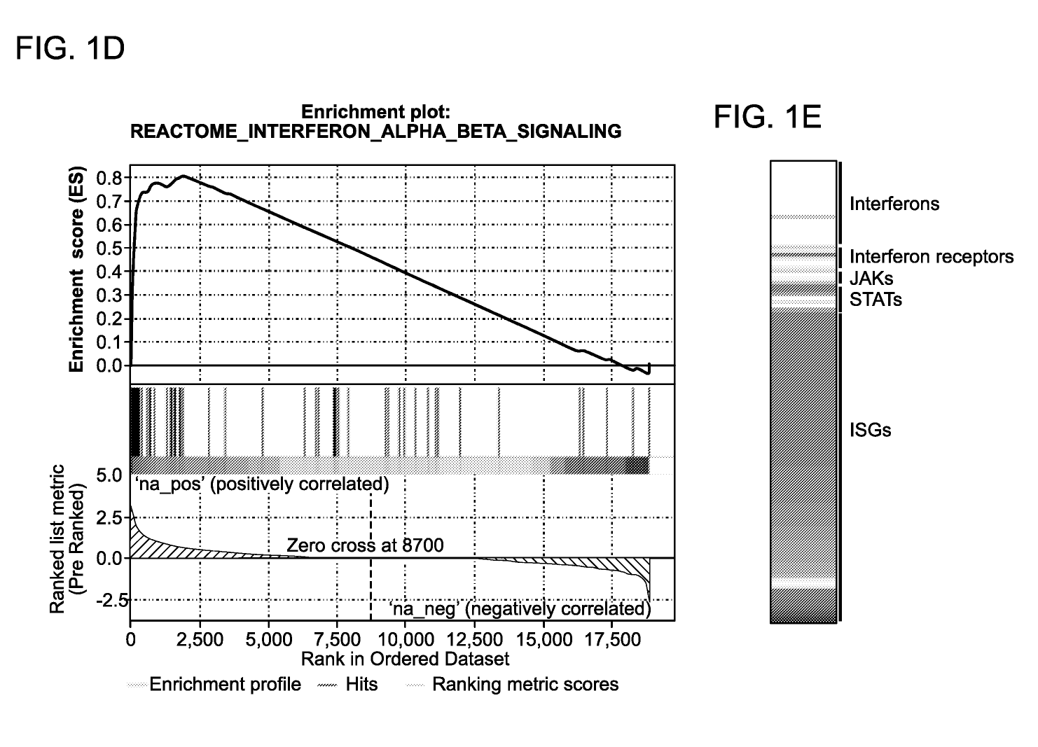

EZH2 mutant-containing DLBCL cell line KARPAS-422 showed temporal sensitivity to treatment with small the molecule inhibitor of EZH2, Inhibitor 1. See FIG. 1a. Cells were treated with various concentrations of Inhibitor 1, with cell viability monitored at day 4, day 8, and day 12 of treatment. The number of viable cells (% viable) was normalized to corresponding DMSO control at each time point, ±SEM (n=3). H3K27me3 and total H3 was monitored via MSD ELISA at day 4, 7, and 11. See FIG. 1b. Relative H3K27me3 levels were normalized to total H3, then to DMSO treated control cells, ±SEM (n=3). A heatmap representation of genes differentially expressed in KARPAS-422 cells treated with 0.1% DMSO or 1.5 μM Inhibitor 1 for 4 days, then subjected to RNA-sequencing. See FIG. 1c. Heatmap displays all genes where log2 fold change is greater than or equal to 1, and p<0.05 when comparing the average of 3 Inhibitor 1 treated replicates to 3 DMSO treated replicates. Genes were sorted from highest average log2 fold change in Inhibitor 1 treated samples to lowest. FIG. 1d is a gene set enrichment analysis barcode plot of one type I interferon pathway gene set showing significant enrichment of genes identified in KARPAS-422 cell from RNA-sequencing dataset shown in FIG. 1c. A heatmap representation of differential expression of gene groups within the interferon signaling pathway in KARPAS-422 cells from RNA-sequencing dataset in FIG. 1c. See FIG. 1e. All interferon, interferon receptor, JAK family kinase (JAKs), and STAT genes (STATs) are represented in heatmap, with a subset of JAKs and STATs showing log2 fold change greater than 1 when comparing Inhibitor 1 treated cells to DMSO control. Only the subset of interferon stimulated genes (ISGs) showing log2 fold change greater than or equal to 1 are represented in the heatmap.

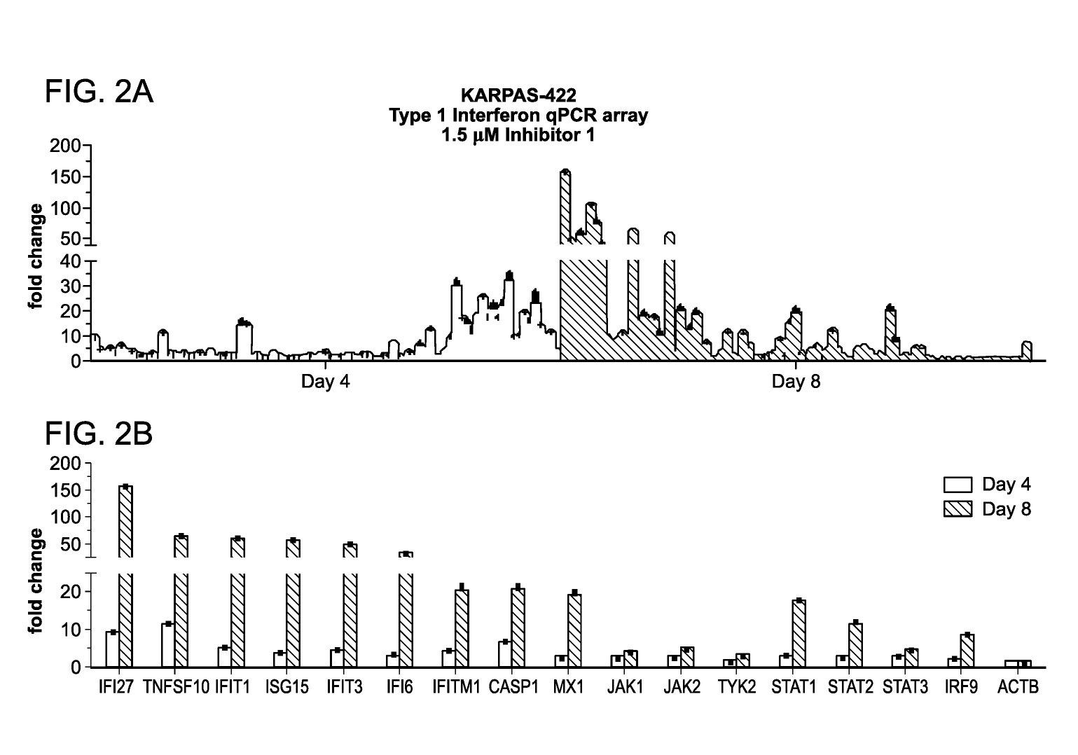

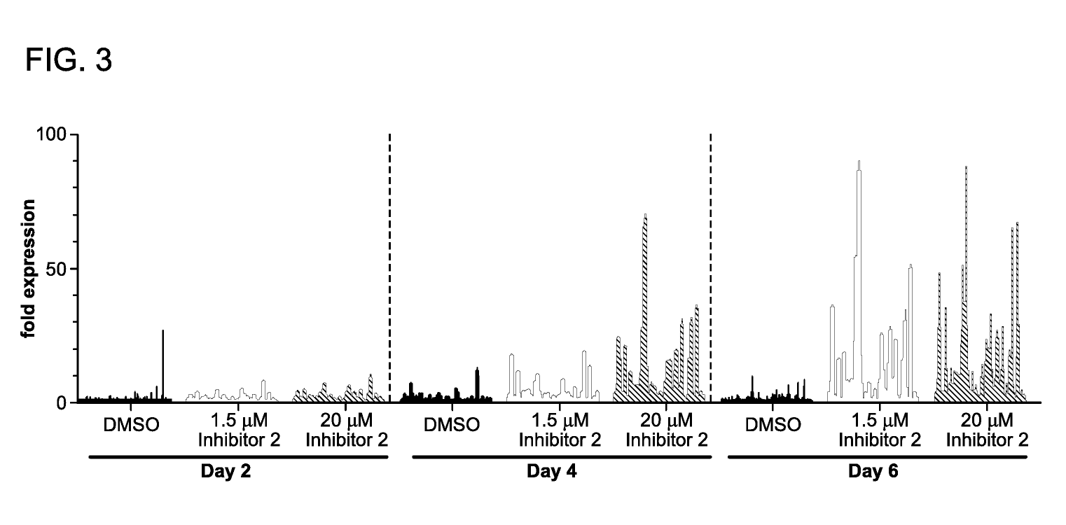

KARPAS-422 cells were treated with 0.2% DMSO or 1.5 or 20 μM EZH2 inhibitor Inhibitor 2 for 6 days total, with cells harvested at days 2, 4, and 6 for RNA extraction. See FIG. 2a. RNA was converted to cDNA, and analyzed via qPCR using a type I interferon gene specific qPCR array. Gene expression was normalized to the geometric mean of 5 genes of reference at each time point for each treatment, then Inhibitor 2 treated samples compared to DMSO treated samples to generate fold change values for each time point. Fold change for each gene is represented as a single bar in graph, with relative position of each gene remaining constant between days and treatment groups. Fold change +/−SEM, n=2. KARPAS-422 cells were treated with 0.1% DMSO or 1.5 μM EZH2 inhibitor Inhibitor 1 for 8 days total, with cells split and drug re-applied at day 4. RNA was harvested at days 4 and 8, converted to cDNA, and analyzed via qPCR using a type I interferon gene specific qPCR array. See FIG. 2b. Gene expression was normalized to the geometric mean of 5 genes of reference at each time point for each treatment, then Inhibitor 1 treated samples compared to DMSO treated samples to generate fold change values for each time point. Day 4 data shown in gray, day 8 in blue. Fold change for each gene is represented as a single bar in graph, with relative position of each gene remaining constant between day 4 and day 8. Fold change+/−SEM, n=2. FIG. 3 shows gene expression changes upon Inhibitor 1 treatment for genes showing highest fold change, and other genes of interest (JAKs and STATs), from FIG. 2b are visualized. Day 4 fold change in gray, day 8 in blue. Fold change+/−SEM, n=2.

### Synergism between EZH2 Inhibitors and Type I Interferons

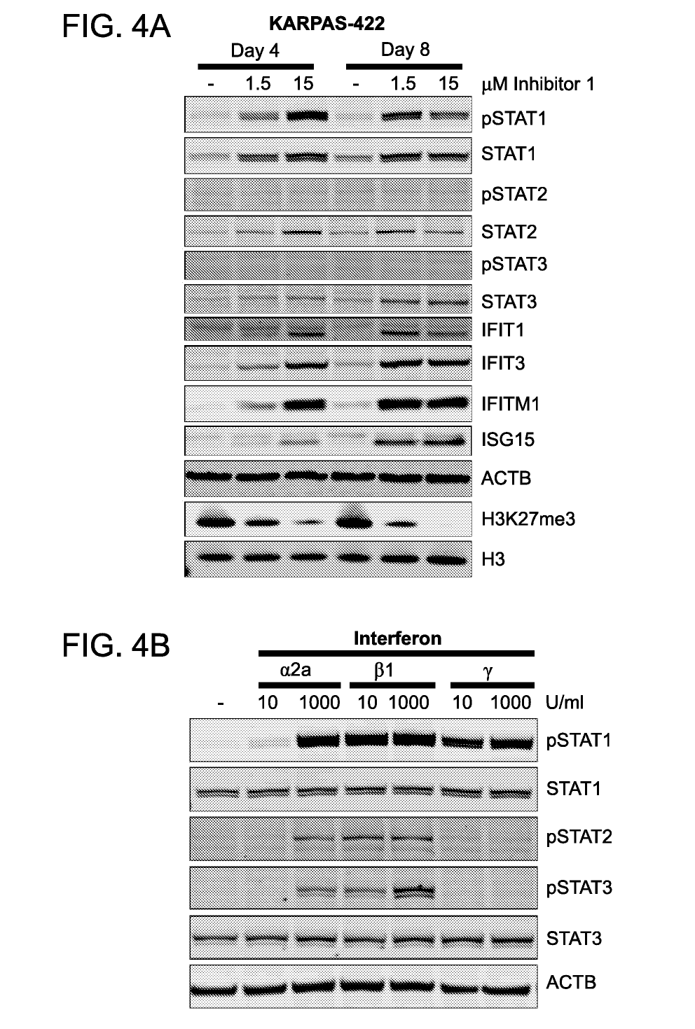

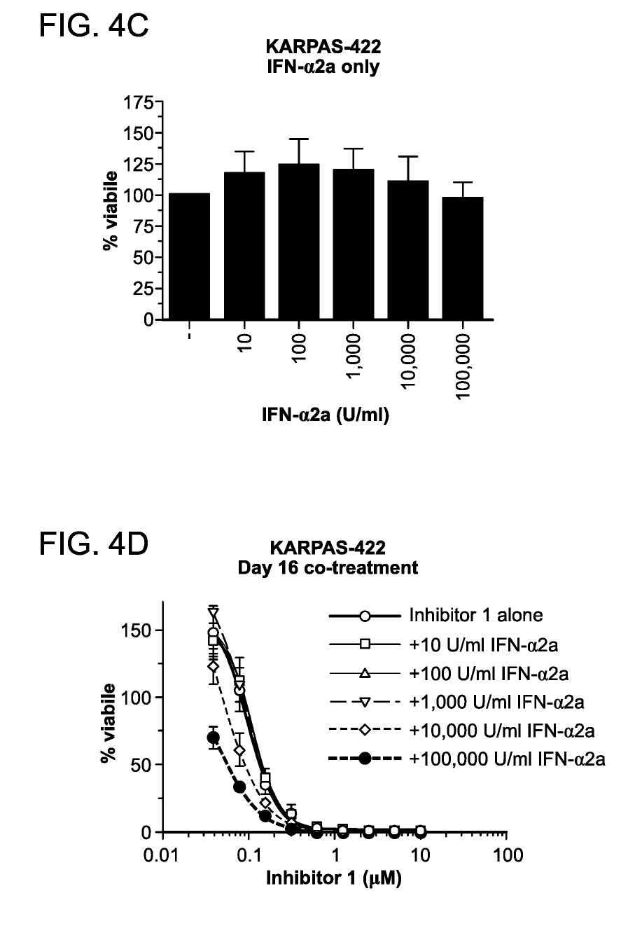

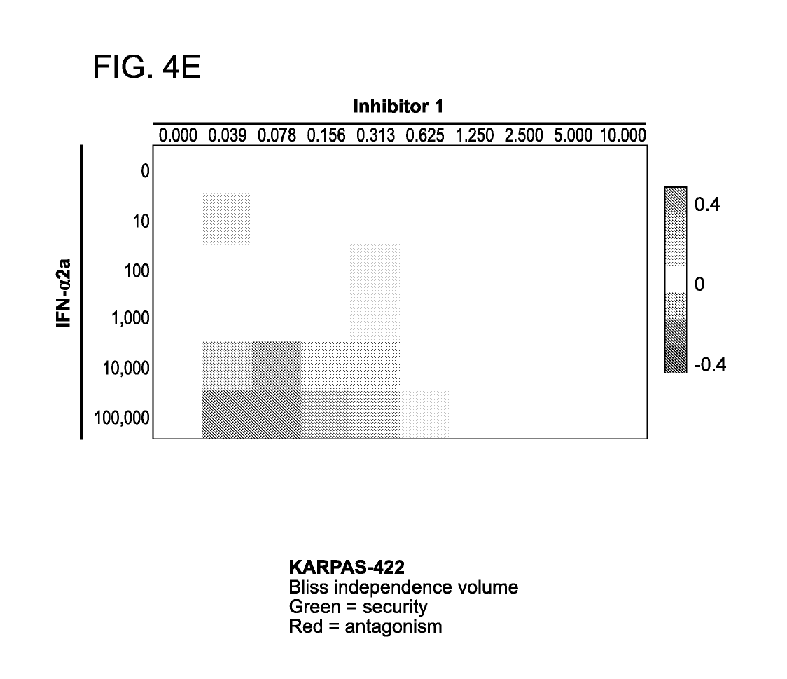

KARPAS-422 cells treated with 0.15% DMSO, 1.5 or 15 μM Inhibitor 1 for 8 days total, with reseeding and fresh compound addition at day 4. See FIG. 4a. Samples harvested at day 4 and 8, and analyzed via western blot with antibodies against the indicated proteins. FIG. 4b shows KARPAS-422 cells treated with 0.1% BSA control, or 10 or 1000 U/ml interferon α2a, β1, or γ for 1 hour before harvesting. Samples analyzed via western blot with antibodies against the indicated proteins. FIG. 4c and FIG. 4d show KARPAS-422 cells were co-treated with a titrations of Inhibitor 1 and interferon (IFN) α2a for 16 days total, with re-seeding and fresh application of both drugs at days 4, 8, and 12. Single drug sensitivities were calculated by incubation with drug of interest and diluent for other drug (i.e. 0.1% DMSO for Inhibitor 1, or 0.1% BSA for IFN). Cell viability measurements were taken using Cell Titer Glo, and % viable calculated by normalizing luminescence values to DMSO+0.1% BSA control treated cells. c displays day 16 single agent sensitivity to IFN α2a, and d displays day 16 single agent sensitivity to Inhibitor 1 alone, and in the presence of a titration of IFN α2a. % viable+/−SEM, n=3. For each combination of drug concentrations, a Bliss independence volume score was calculated by comparing the experimental cell viabliltiy to predicted cell viability based on single agent sensitivities. See FIG. 4e. Bliss values are represented as a heatmap, with green indicating synergy and red indicating antagonism between the two drugs.

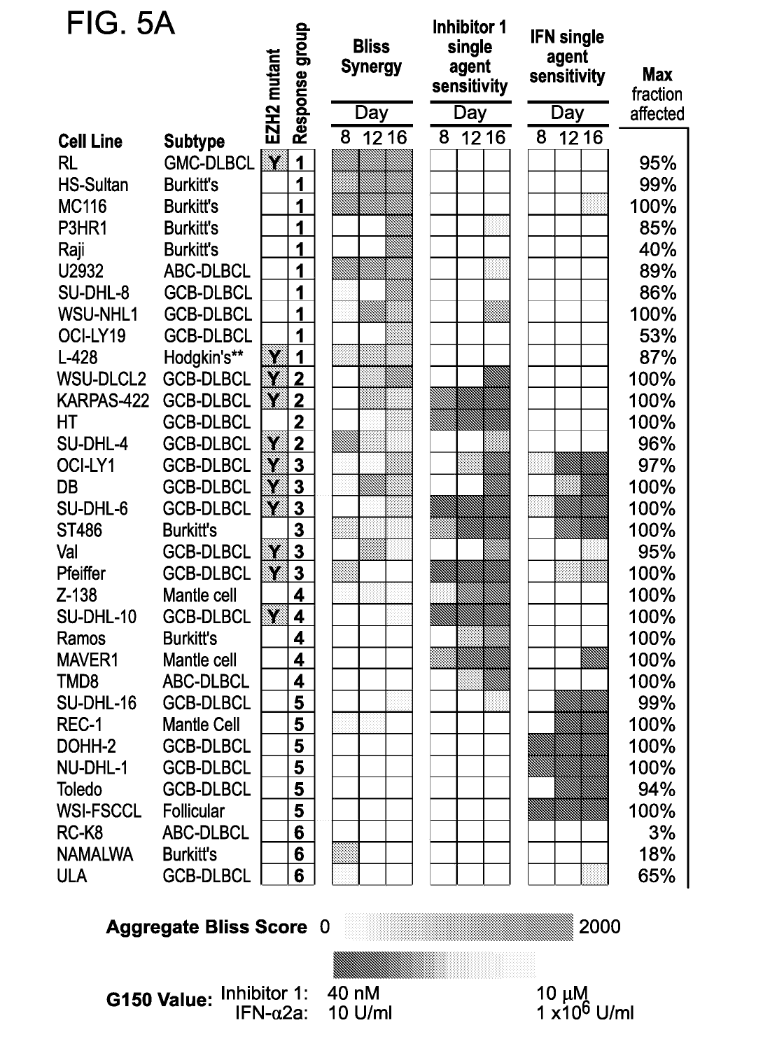

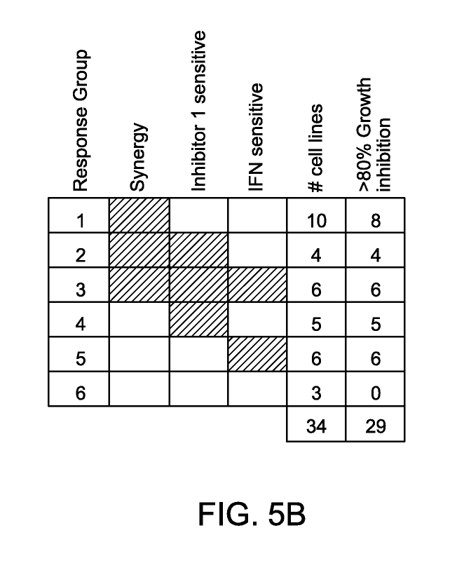

Cell models of non-Hodgkin's lymphoma (NHL) were treated with titrations of both Inhibitor 1 and interferon α2a for 16 days total. See FIG. 5a. Cells were reseeded, with fresh application of drug at day 4, 8, and 12. Cell viability was measured via Cell Titer Glo at days 8, 12, and 16 overall. Most cell lines were treated with only Inhibitor 1 for the first 4 days, before co-treatment with Inhibitor 1 and IFN starting at day 4 until day 16. In cell lines that show a rapid response to Inhibitor 1 alone, cells were co-treated with both drugs starting at day 0. % viability was determined comparing luminescence values for each combination to a 0.1% DMSO+0.1% BSA control. For each combination of drug concentrations, a Bliss independence volume score was calculated by comparing the experimental cell viability to predicted cell viability based on single agent sensitivities. The sum of Bliss volumes for all combinations tested for a given cell line was calculated, and represented as an aggregate Bliss score. The table displays the following values: cell line name; NHL subtype (GCB-DLBCL=germinal center B-cell like diffuse large B cell lymphoma; Burkitt's lymphoma; ABC-DLBC=activated B cell-like diffuse large B cell lymphoma; Mantle cell lymphoma; Follicular lymphoma; Burkitt's lymphoma; and ** L-428 is indicated as a Hodgkin's lymphoma in published literature, however, given it harbors an activating EZH2 mutation, it is most likely mis-categorized, and is instead a GCB-DLBCL cell line); EZH2 mutant, where Y=presence of activating mutation in SET domain of EZH2; response group, explained in FIG. 5b; Bliss synergy scores calculated for days 8, 12, and 16, with darkest green shading indicating highest aggregate score; Single agent sensitivity to Inhibitor 1 at days 8, 12, and 16, with heatmap representing GI₅₀ values (min=40 nM, max=10 μM); Single agent sensitivity to IFN α2a at days 8, 12, and 16, with heatmap representing GI₅₀ values (min=10 U/ml, max=1×10⁶ U/ml); and Max fraction affected=highest % of cell growth inhibition observed across all tested combination of drug concentrations for a given cell line. Cell lines are sorted by response group, then by day 16 aggregate Bliss score. Cell lines were divided into 6 response groups. 1=cell lines insensitive to both Inhibitor 1 and IFN single agent treatments, but show synergistic cell growth inhibition upon treatment with the combination of both drugs; 2=Cell lines sensitive to Inhibitor 1 single agent treatment, insensitive to IFN single agent treatment, but show synergistic cell growth inhibition to combination; 3=Cell lines sensitivity to both Inhibitor 1 and IFN single agent treatments, and show synergy to combination; 4=Cell lines sensitive to Inhibitor 1 single agent treatment, but are insensitive to IFN single agent treatment and show no synergy when treated with a combination of both treatments; 5=Cell lines sensitive to IFN single agent treatment, but are insensitive to Inhibitor 1 single agent treatment and show no synergy when treated with combination of both treatments; 6=Cell lines insensitive to both single agent Inhibitor 1 and IFN, and show no synergy to combination. See FIG. 5b. 29 out of 34 cell lines (85%) show >80% cell growth inhibition to either single agent Inhibitor 1 or IFN treatment, or the combination of both treatments.

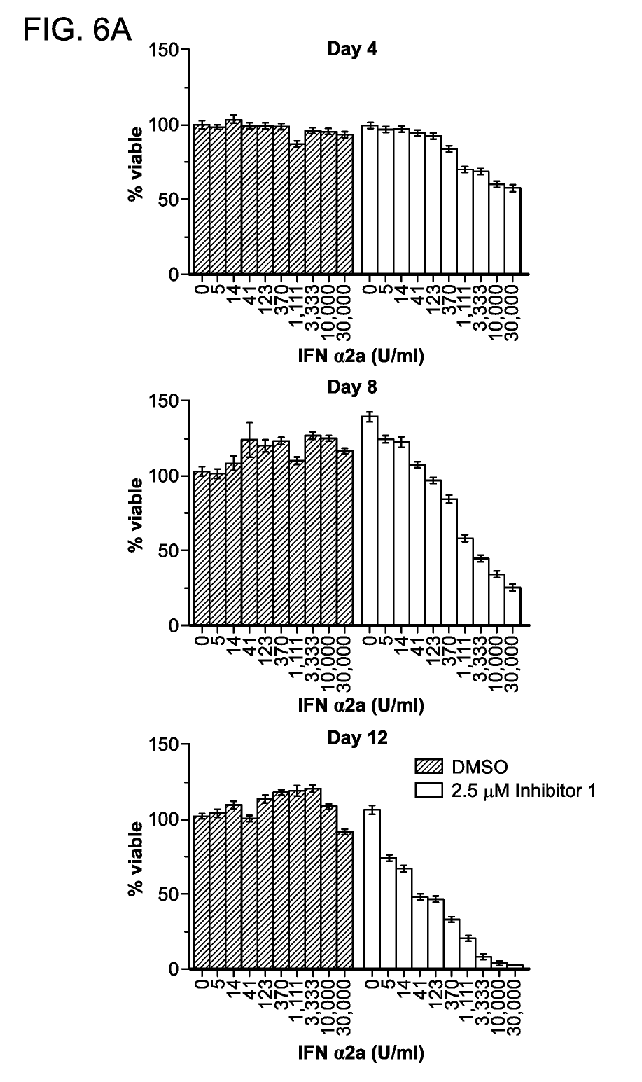

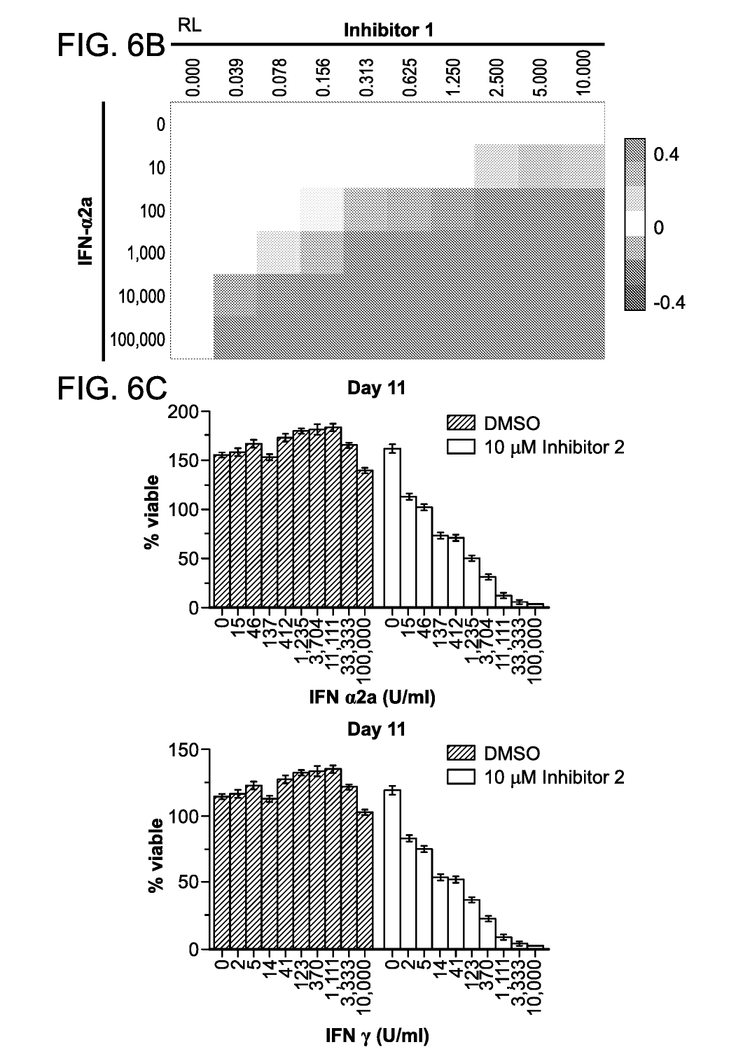

### Interferon Response(s) Only Elicited by Combination of EZH2 Inhibitors and Type I Interferons Shown by FIG. 6

In addition to the synergistic effect on therapeutic activity now found and described between EZH2 inhibitors and type I interferons, it was further unexpectedly found that certain interferon responses were only elicited by the combination of an EZH2 inhibitor and a type I interferon, and not by each of these agents alone. That is, new pathways were targeted, which only resulted from treatment with the combination of an EZH2 inhibitor and a type I interferon. See e.g., FIG. 6 described below.

RL cells were pre-treated with 0.1% DMSO or 2.5 μM Inhibitor 1 for 4 days, then re-seeded and treated with a titration of IFN α2a, while maintaining DMSO or Inhibitor 1 treatment. See FIG. 6a. Cells were co-treated as such for an additional 12 days, with reseeding and fresh application of both drugs every 4 days. Cell viability measurements were taken every 4 days using Cell Titer Glo, and % viable calculated by normalizing luminescence values to DMSO+0.1% BSA treated control cells at each time point. A separate experiment, RL cells were treated with titrations of both Inhibitor 1 and interferon α2a for 16 days total. See FIG. 6b. Cells were re-seeded, with fresh application of drug at days 4, 8, and 12. Cell viability was measured via Cell Titer Glo at days 8, 12, and 16 overall. Cells were treated with only Inhibitor 1 for the first 4 days, before co-treatment with Inhibitor 1 and IFN starting at day 4 until day 16. % viability was determined comparing luminescence values for each combination to a 0.1% DMSO+0.1% BSA control. For each combination of drug concentrations, a Bliss independence volume score was calculated by comparing the experimental cell viability to predicted cell viability based on single agent sensitivities. Bliss values for day 16 are represented as a heatmap, with green indicating synergy and red indicating antagonism between the two drugs. Similar to a, RL cells were pre-treated with 0.1% DMSO or 10 μM Inhibitor 2 for 4 days, then reseeded and treated with a titration of either IFN α2a or IFN γ, while maintaining DMSO or Inhibitor 1 treatment. See FIG. 6c. Cells were co-treated as such for an additional 11 days, with reseeding and fresh application of both drugs every 3-4 days. Cell viability measurements were taken every 4 days using Cell Titer Glo, and % viable calculated by normalizing luminescence values to DMSO+0.1% BSA treated control cells at each time point. Given that the RL lymphoma cell line was insensitive to treatment with either an EZH2 inhibitor or IFN alone, but the combination of both modalities induced potent dose-dependent cell growth inhibition suggests the combination uniquely perturbs a molecular pathway that neither modality is able to sufficiently perturb on its own. This may occur via modulation of a novel molecular regulatory cue neither modality was previously known to affect, or via an amplified modulation of a pathway previously linked to one modality, but not the other, such that the combination uniquely affects the pathway triggering potent cell growth inhibition.

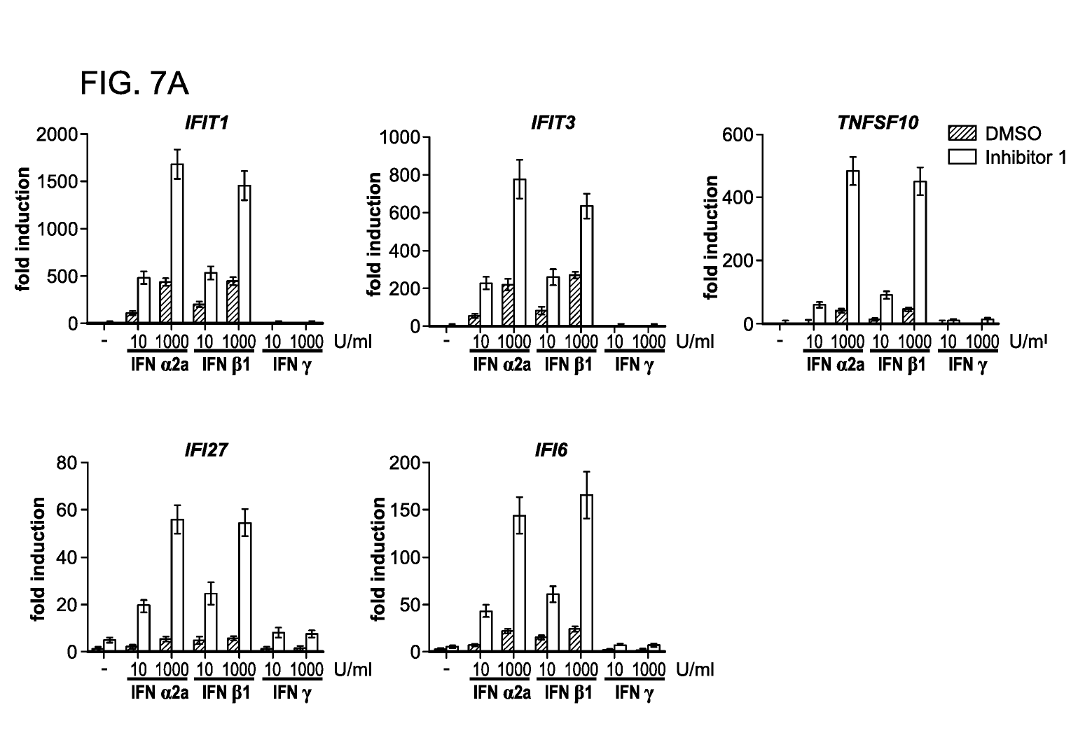

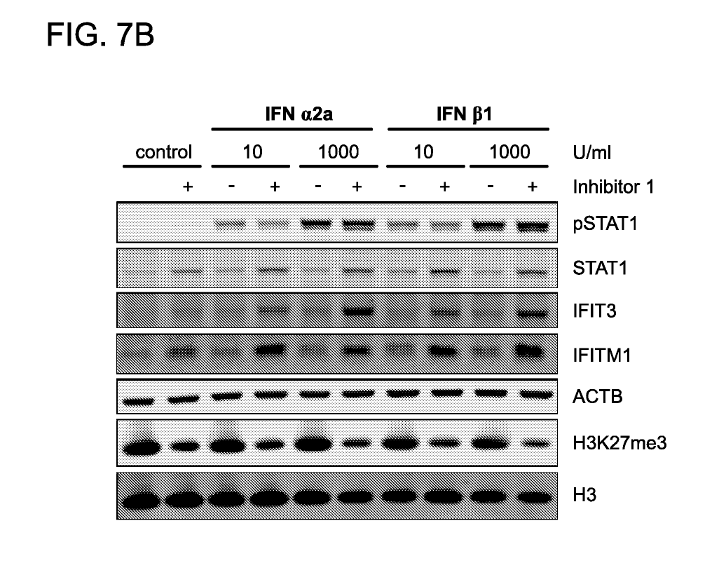

RL cells were pre-treated with 0.1% DMSO or 1.5 μM Inhibitor 1 for 4 days before application of 0.1% BSA, or 10 or 1000 U/ml IFN α2a, β1, or γ for 4 additional hours. RNA was extracted, converted to cDNA, and qPCR performed for indicated genes and a gene of reference (ACTB or TBP). See FIG. 7a. Fold induction values determined by normalizing gene of interest expression value to gene of reference, then comparing to DMSO+BSA control, +/−SEM, n=4. RL cells were pre-teated as above, then treated with 0.1% BSA, or 10 or 1000 U/ml IFN α2a or β1 for 4 additional hours. Protein lysates were generated, and analyzed via western blot with antibodies against the proteins indicated. See FIG. 7b.

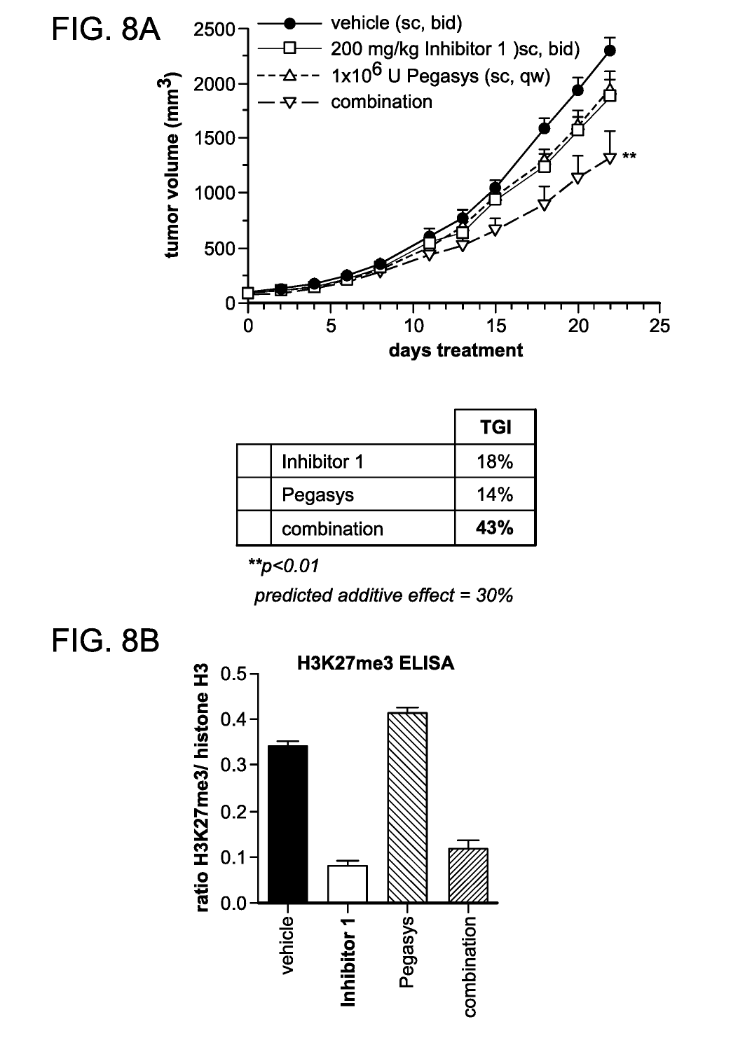

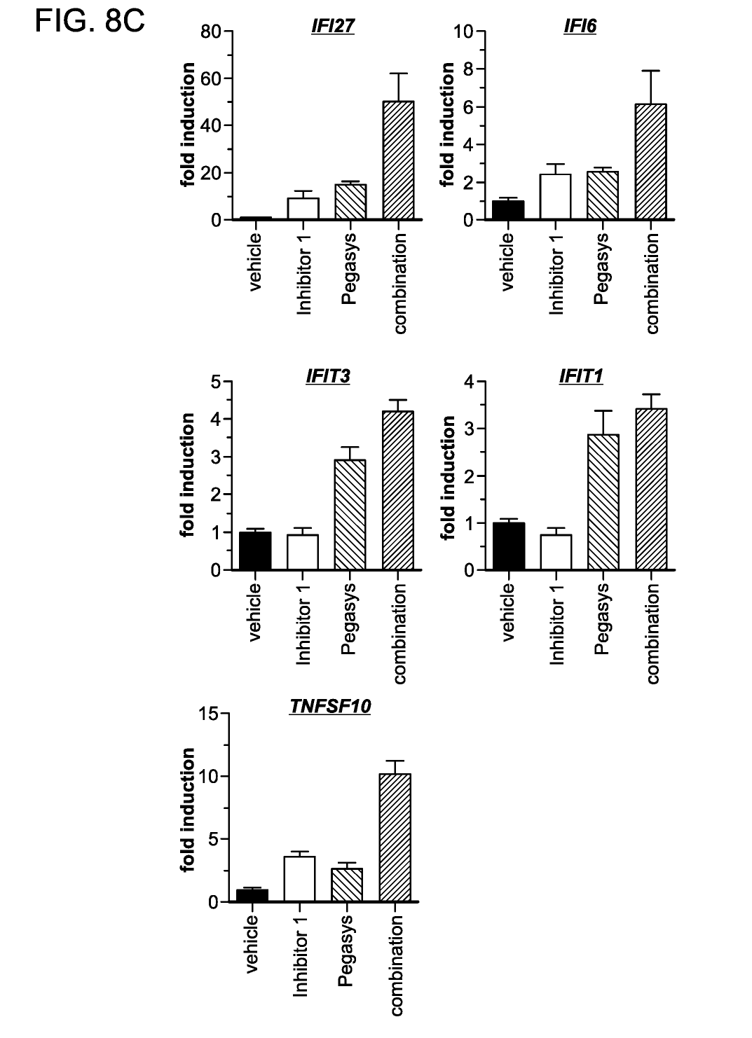

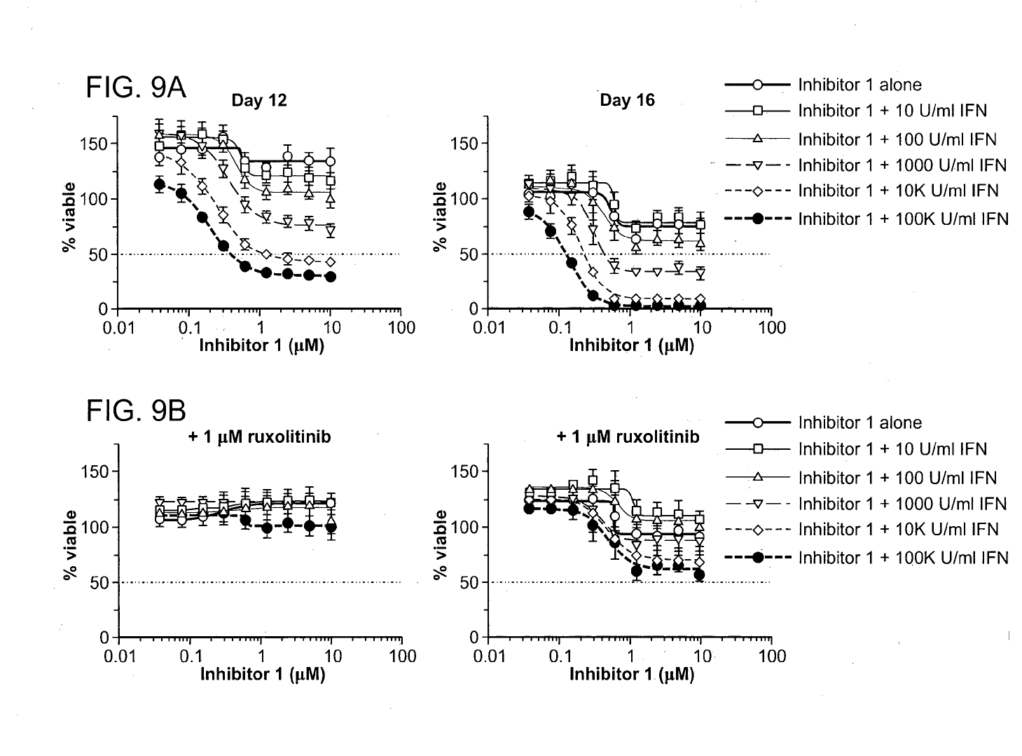

The data in FIG. 8a shows SCID mice with RL xenograft were treated with vehicle, 200 mg/kg Inhibitor 1, sc, bid, 1×10⁶ units peginterferon alfa-2a, sc, qw, or a combination of both treatments, and tumor volume measurements recorded at indicated time points. Tumor growth inhibition (TGI) values were calculated, and a one-way ANOVA analysis performed across all groups, with a post-hoc Tukey test to compare groups. The combination treatment was statistically significant when compared to vehicle control, p<0.01. The predicted additive tumor growth inhibition of the combination treatment from this study was 30%, whereas the actual TGI value for the combination was 43%, suggesting a synergistic response. Palpable tumors were harvested on final day of study, homogenized, and analyzed for H3K27me3 and total H3 content using MSD ELISA. H3K27me3 values were normalized to total H3, +/−SEM, n=3. See FIG. 8b. In parallel, tumors were homogenized, RNA extracted, converted to cDNA, and qPCR was performed on selected ISGs and a gene of reference (GOR). Expression values were normalized to the gene of reference for each animal, then to the vehicle control to determine fold induction, +/−SEM, n=4. See FIG. 8c.

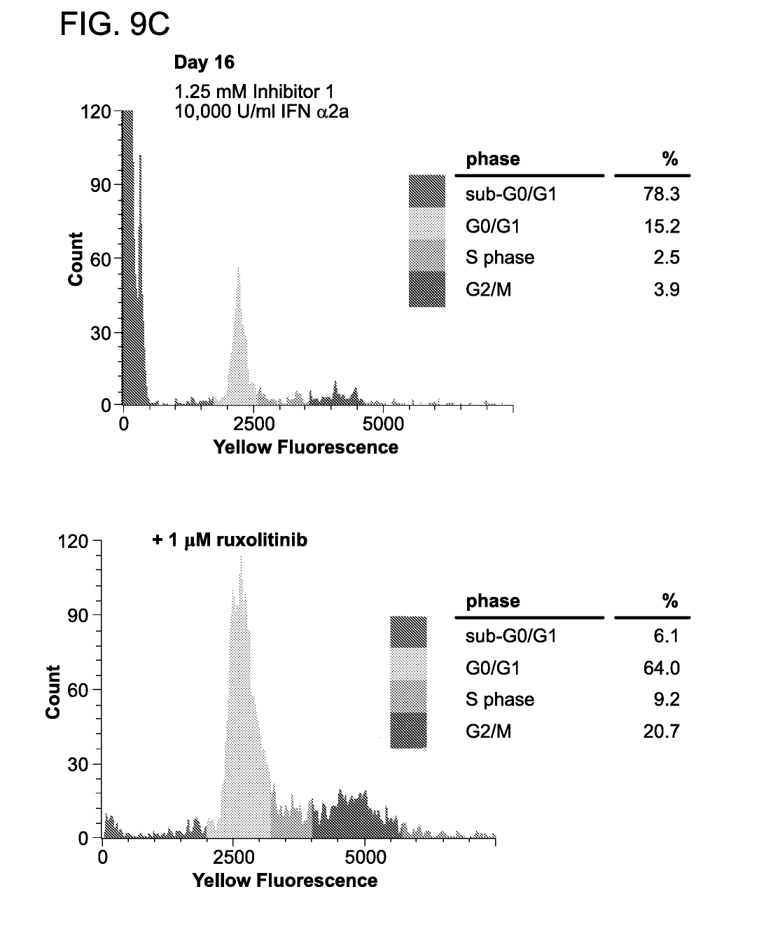

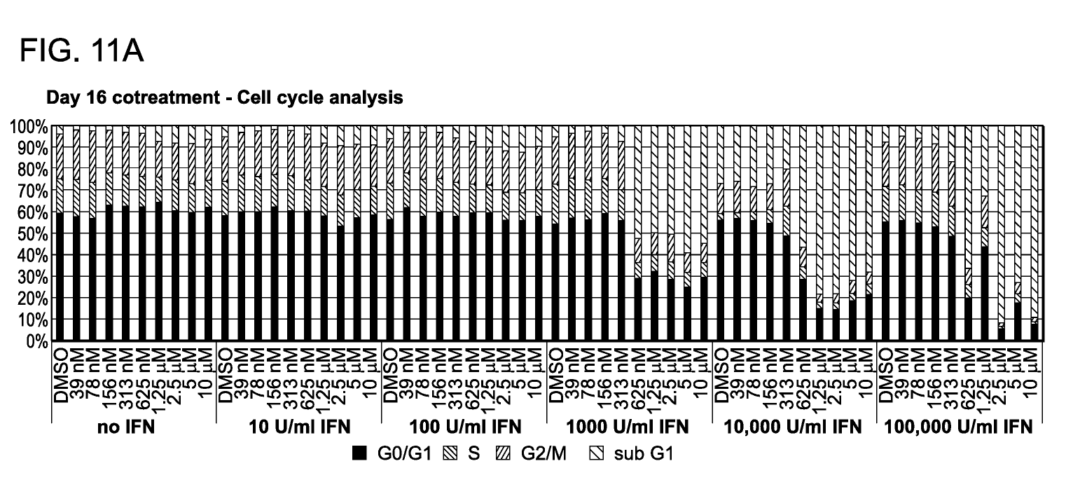

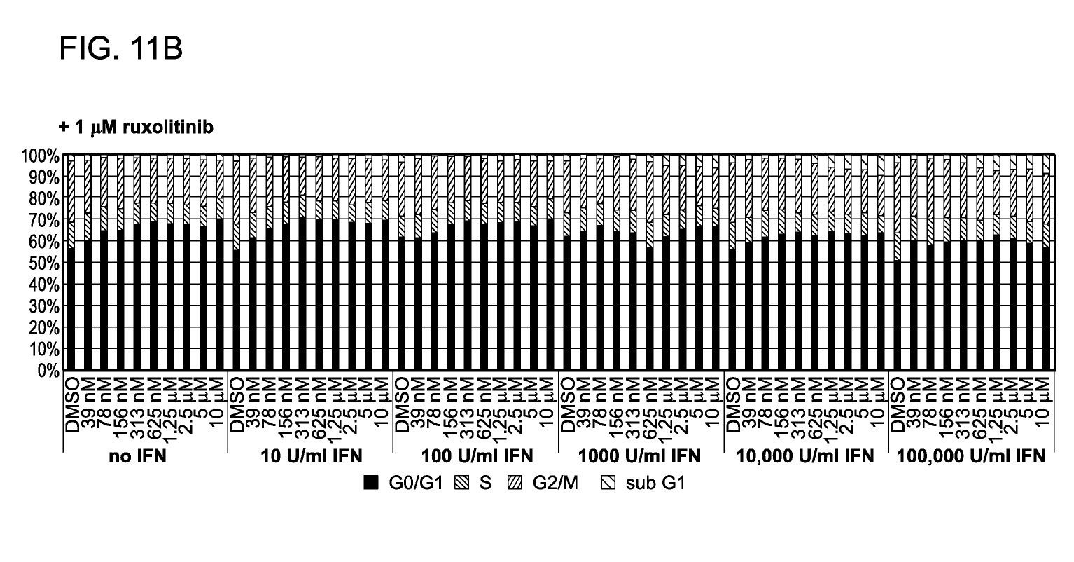

RL cells were co-treated with a titration of both Inhibitor 1 and IFN α2a for 16 days total, with re-seeding and fresh application of both drugs at days 4, 8, and 12. FIG. 9a. Cell viability measurements were taken via Cell Titer Glo at days 8, 12, and 16. % viability was determined comparing luminescence values for each combination to a 0.1% DMSO+0.1% BSA control. Data from day 12 and 16 are displayed. In parallel, cells were treated as above, with the addition of 1 μM ruxolitinib to all wells. See FIG. 9b. Data from day 12 and 16 are displayed. For experiments performed in both FIG. 9a and FIG. 9b, an aliquot of cells were also processed for cell cycle analysis. See FIG. 9c. Cells were fixed, then stained with propidium iodide, and DNA content measured with a Guava cell analyzer. Displaying day 16 data for combination of 1.25 μM Inhibitor 1 and 10,000 U/ml IFN α2a+/−1 μM ruxolitinib. % of cells in each phase of cell cycle determined from cell count quantitation from applied gates, as indicated by color. See FIG. 11 for quantitation of full titration of Inhibitor 1 and IFN α2a.

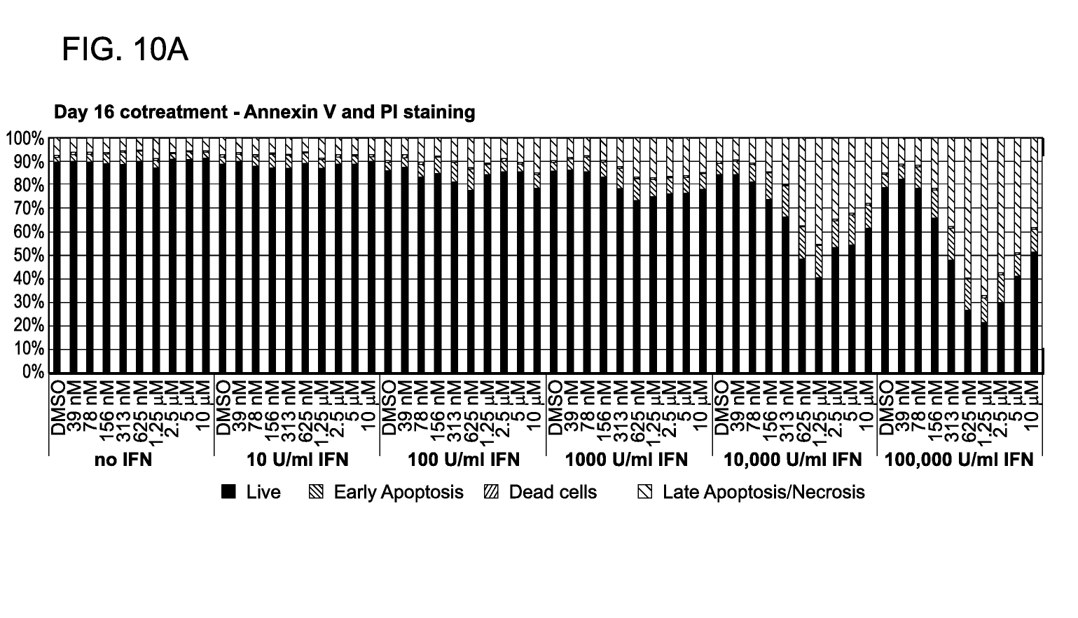

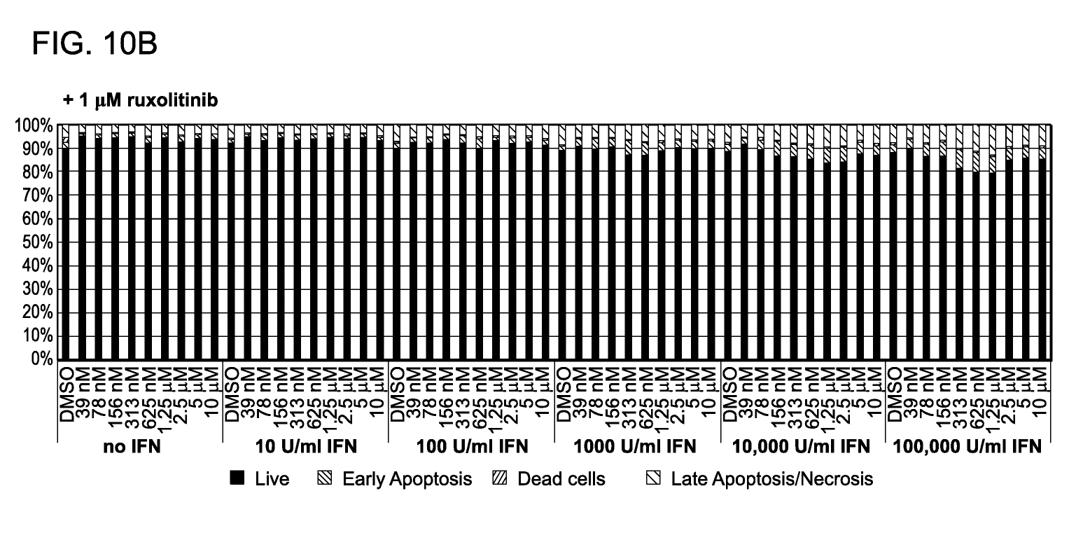

For experiments described above and data illustrated in FIG. 9, an aliquot of cells were processed via Annexin V and propidium iodide staining, then quantitated with a Guava cell analyzer. See FIG. 10a and FIG. 10b. Live cells=Annexin and PI negative; Early apoptosis=Annexin positive, PI negative; Late apoptosis/Necrosis=Annexin and PI positive; Dead cells=Annexin negative, PI positive. Data displayed for full titration of Inhibitor 1 and IFN α2a+/−1 μM ruxolitinib at day 16 overall. See FIG. 11a and FIG. 11b. For experiments described in FIG. 9, an aliquot of cells were processed for cell cycle by fixation, then propidium iodide staining, then quantitated with a Guava cell analyzer. Data displayed for full titration of Inhibitor 1 and IFN α2a+/−1 μM ruxolitinib at day 16 overall.

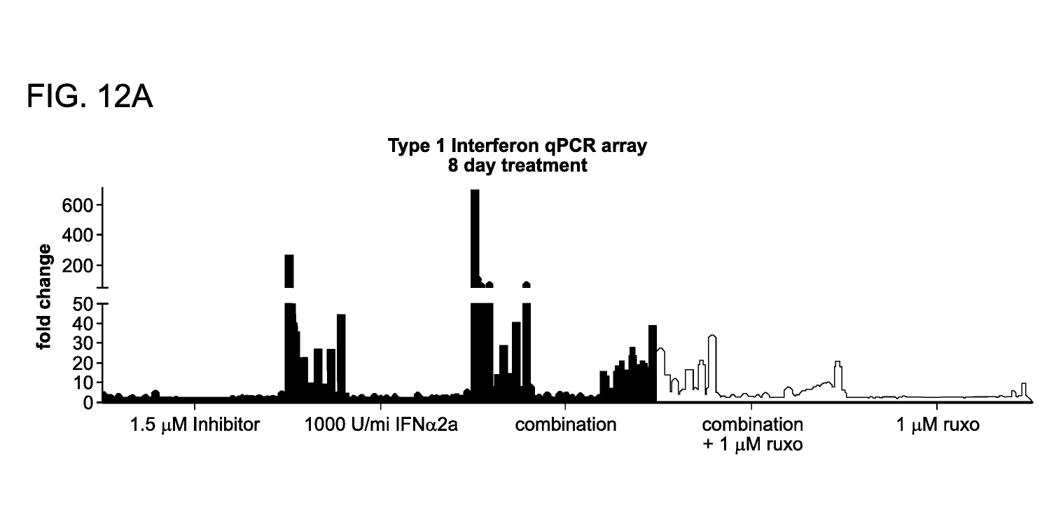

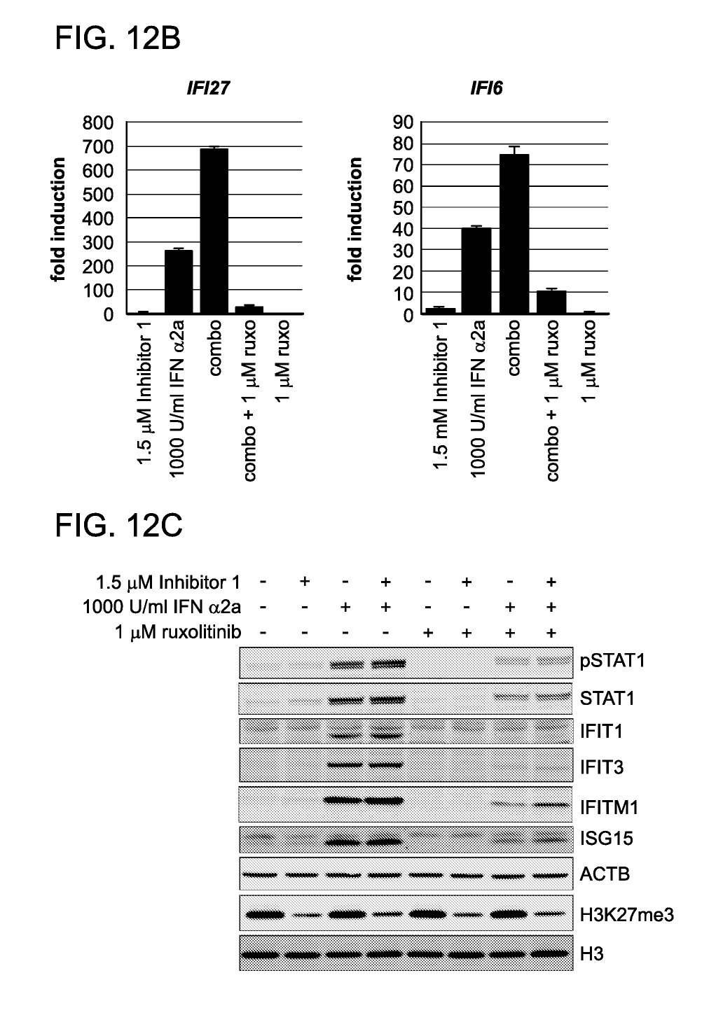

For the data shown in FIG. 12a RL cells were treated with 0.1% DMSO, 1.5 μM Inhibitor 1, 1000 U/ml IFN α2a, the combination of both, 1 μM ruxolinitib, or the combination of all three for 8 days total, with re-seeding and application of fresh drug at day 4. RNA was harvested at days 4 and 8, converted to cDNA, and analyzed via qPCR using a type I interferon gene specific qPCR array. Gene expression was normalized to the geometric mean of 5 genes of reference at each time point for each treatment, then each treatment compared to DMSO treated samples to generate fold change values. Fold change in the steady state transcript levels for each gene represented as a single bar in graph, with relative position of each gene remaining constant between treatment groups. Fold change+/−SEM, n=2. Representation of IFI27 and IFI6 genes from type I interferon qPCR array from a is shown in FIG. 12b. In parallel to RNA samples generated in protein lysates were generated and analyzed via western blot with antibodies against the proteins indicated is shown in FIG. 12c.

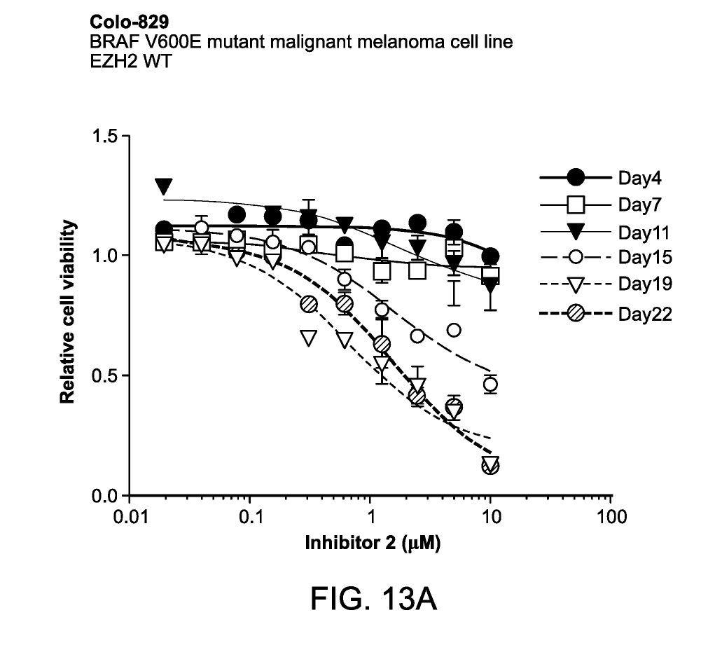

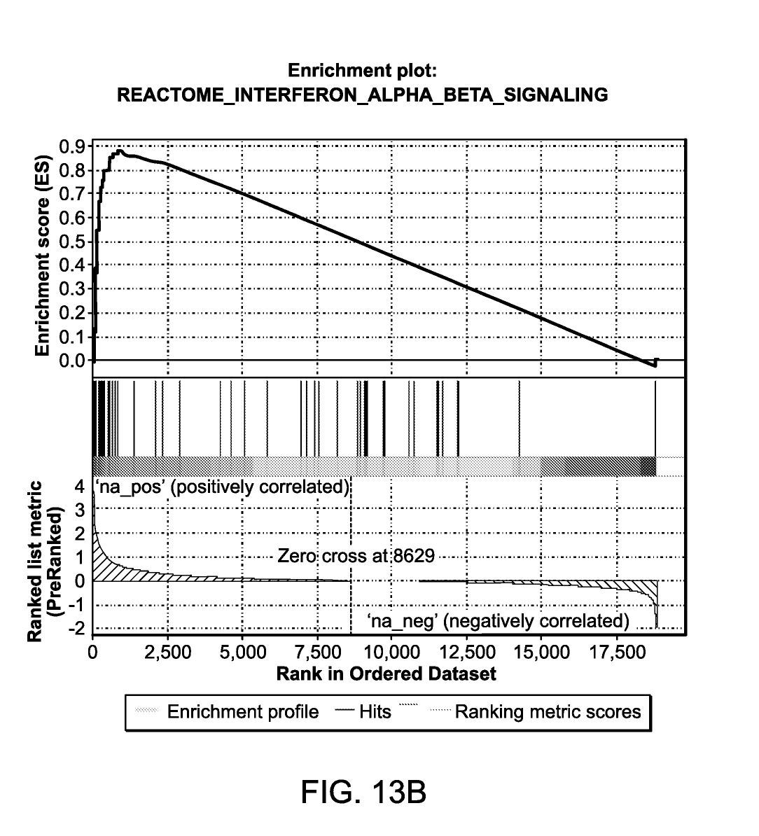

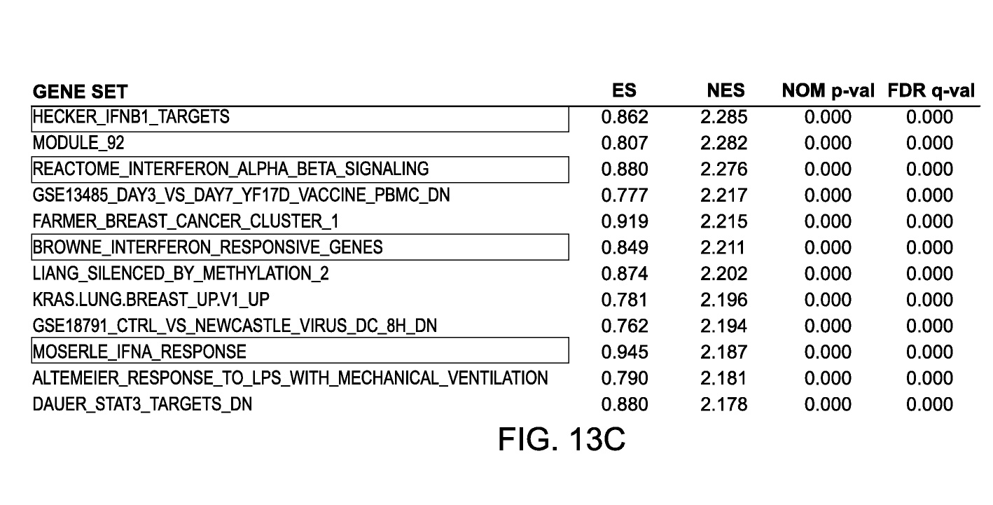

Colo-829 melanoma cells were treated with a titration of Inhibitor 2 for a total of 22 days, with re-seeding and fresh application of drug every 3-4 days. See FIG. 13a. Cell viability was assessed at each splitting event via Cell Titer Glo. Relative cell viability was calculated by comparing luminescence values to that of a DMSO treated control, +/−SEM, n=2. Colo-829 cells were treated with 0.1% DMSO or 10 μM Inhibitor 2 for 8 days, before RNA was extracted and analyzed via RNA-sequencing. See FIG. 13b and FIG. 13c. Differential expression was determined by comparing Inhibitor 2 treated cells to DMSO treated cells. Data was subjected to gene set enrichment analysis using msigdb v4.0 (all categories). Table in c displays gene set, enrichment score (ES), normalized enrichment score (NES), nominal p-value (NOM p-val), and false discovery rate q-value (FDR q-val). FDR<25% used for analysis. Gene sets are sorted by NES. Interferon related gene sets shaded in gray. One of several positively enriched interferon related gene sets displayed in b.

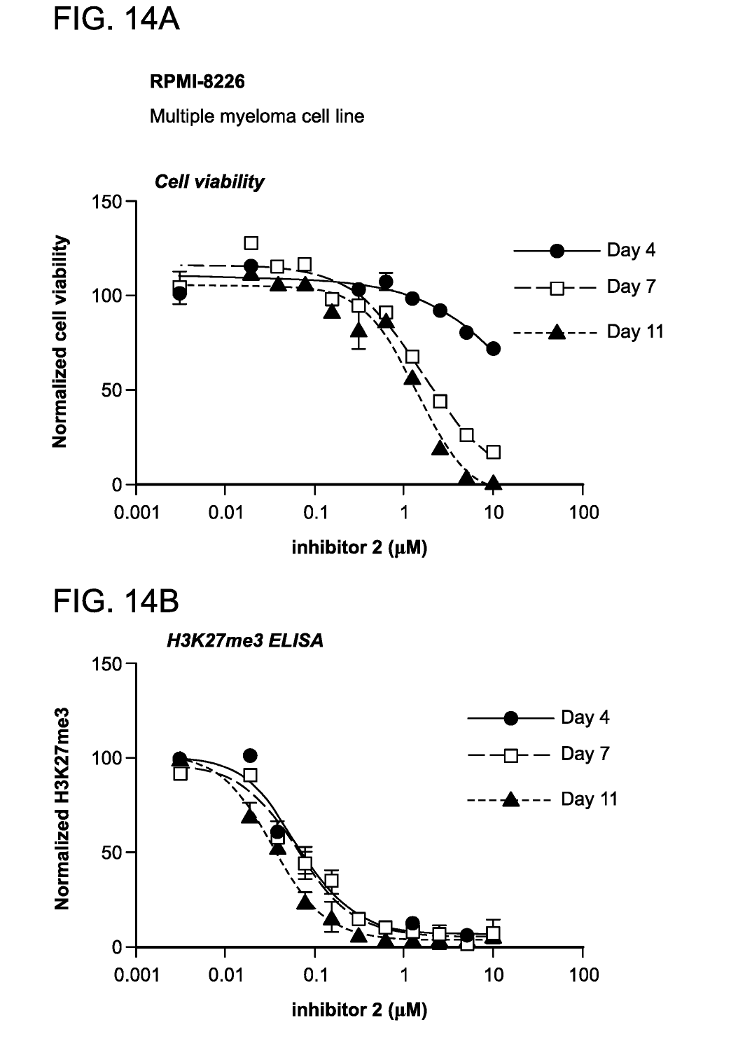

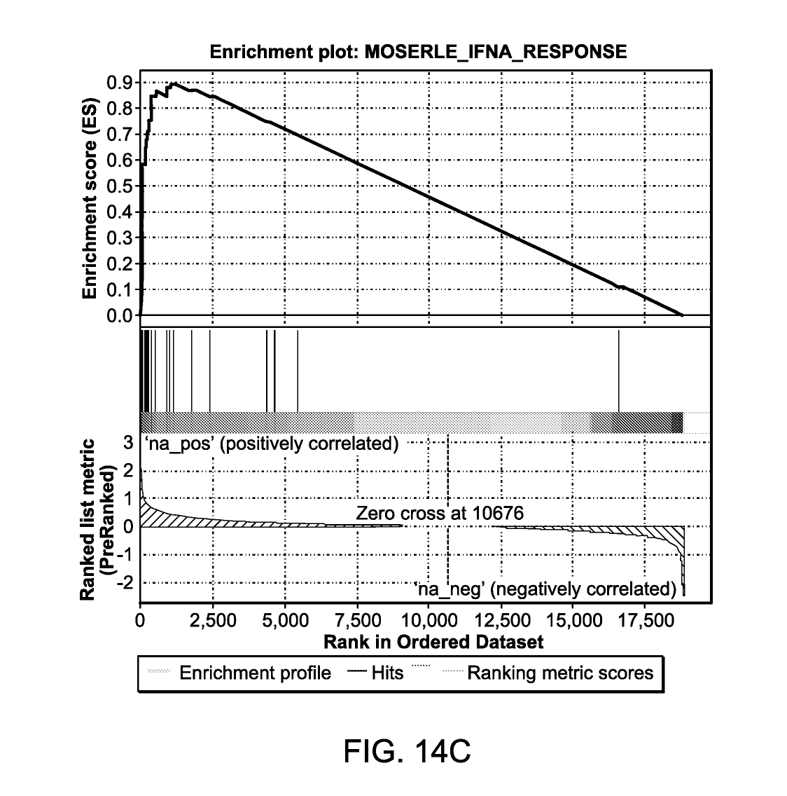

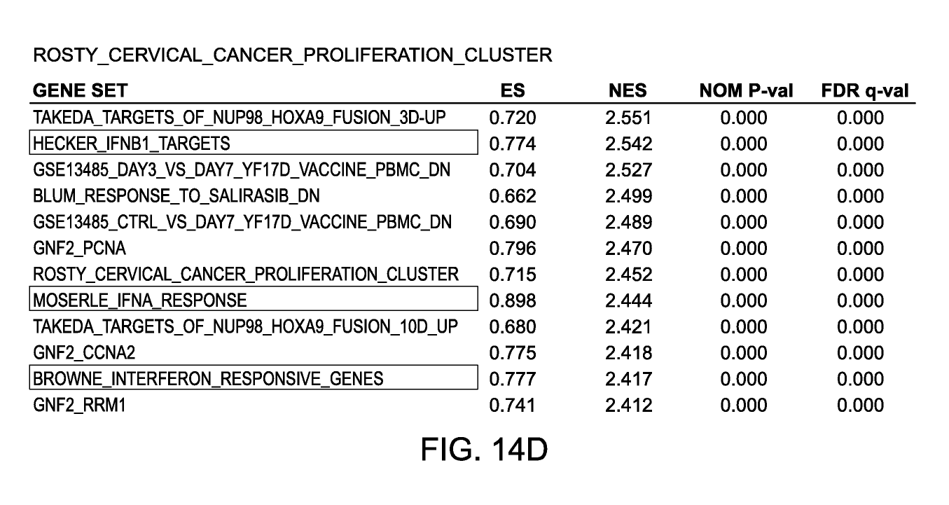

The data in FIG. 14a shows RPMI-8226 multiple myeloma cells treated with a titration of Inhibitor 2 for a total of 11 days, with re-seeding and fresh application of drug every 3-4 days. Cell viability was assessed at each splitting event via Cell Titer Glo. Relative cell viability was calculated by comparing luminescence values to that of a DMSO treated control, +/−SEM, n=2. In parallel, RPMI-8226 cells were monitored for H3K27me3 and total H3 levels by MSD ELISA. See FIG. 14b. K27me3 was normalized to total H3, then to a DMSO treated control at each time point. Assessments were taken at days 4, 7, and 11. +/−SEM, n=3. RPMI-8226 cells were treated with 0.1% DMSO or 1.5 μM Inhibitor 2 for 8 days, before RNA was extracted and analyzed via RNA-seq. See FIG. 14c and FIG. 14d. Differential expression was determined by comparing Inhibitor 2 treated cells to DMSO treated cells. Data was subjected to gene set enrichment analysis (GSEA) using msigdb v4.0 (all categories). Table in d displays gene set, enrichment score (ES), normalized enrichment score (NES), nominal p-value (NOM p-val), and false discovery rate q-value (FDR q-val). FDR<25% used for analysis. Gene sets are sorted by NES. Interferon related gene sets shaded in gray. One of several positively enriched interferon related gene sets displayed in FIG. 14c.

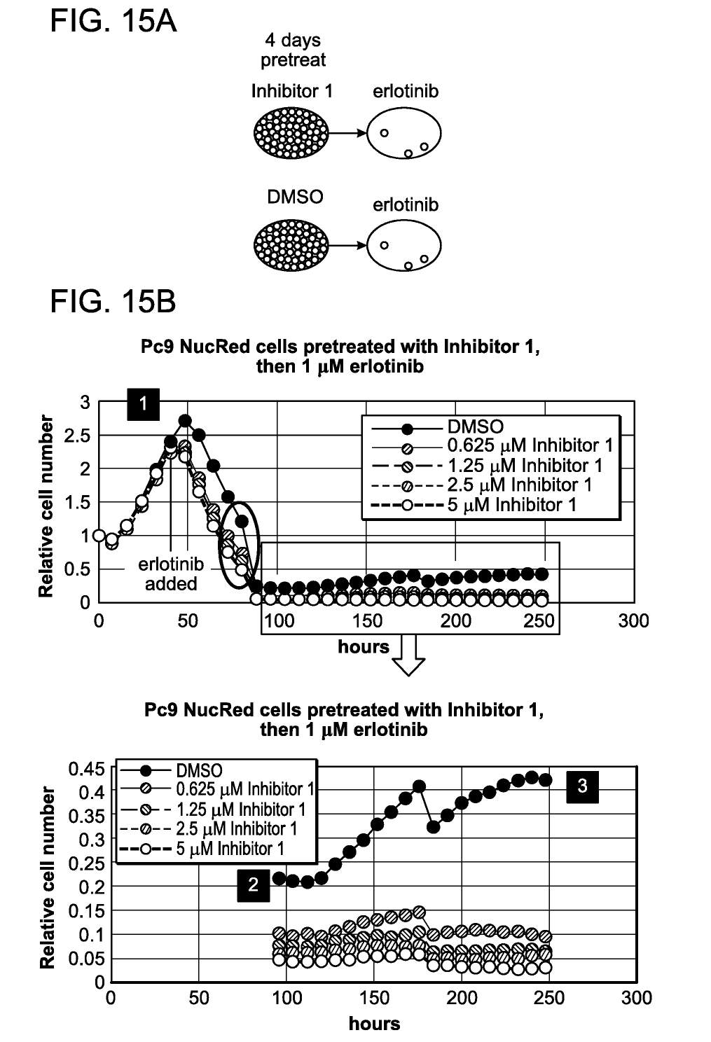

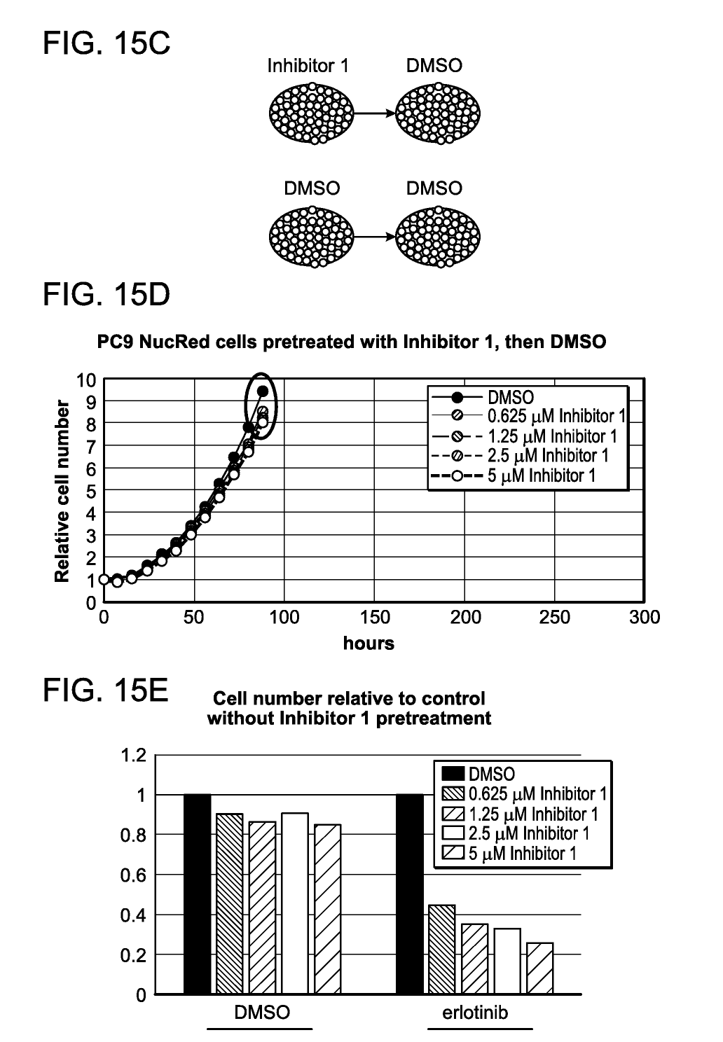

FIG. 15a is a schematic of the experimental design for FIG. 15b, where lung adenocarcinoma PC9 NucRed cells were pre-treated with a titration of Inhbitor 1 for 4 days, before re-seeding. 48 hours later, 1 μM erlotinib was added to the cells, and images were obtained every 12 hours via the Incucyte imaging system. Cells pre-treated with EZH2 inhibitor show (1) faster initiation of cell growth inhibition compared to erlotinib only treated cells, (2) a reduction in the number of drug tolerant persister (DTP) cells remaining after initial erlotinib treatment, and (3) delayed/reduced outgrowth of erlotinib-resistant cells. FIG. 15c is a schematic of experimental design for FIG. 15d and FIG. 15e PC9 NucRed cells were pre-treated with Inhbitor 1 as in FIG. 15a and FIG. 15b, however after 48 hours, were treated with 0.1% DMSO instead of erlotinib. See FIG. 15d. Analysis reveals a 15% reduction in cell number in PC9 cells pretreated with 5 μM Inhbitor 1 compared to DMSO pretreated cells. See FIG. 15e. Comparison of cell number in Inhbitor 1 pre-treated cells compared to DMSO pre-treated cells, then treated acutely with DMSO or erlotinib, as described in FIGS. 15a-d. Acute treatment with DMSO indicated in FIG. 15e corresponds to circled data in FIG. 15d, and acute treatment with erlotinib indicated in FIG. 15e corresponds to circled data in b. 5 μM Inhbitor 1 pretreatment followed by acute DMSO treatment leads to 15% reduction in cell number compared to DMSO pre-treated cells, whereas, 5 μMM Inhbitor 1 pre-treatment followed by acute erlotinib treatment leads to 80% reduction in cell number compared to DMSO pretreatment, acute erlotinib treatment.

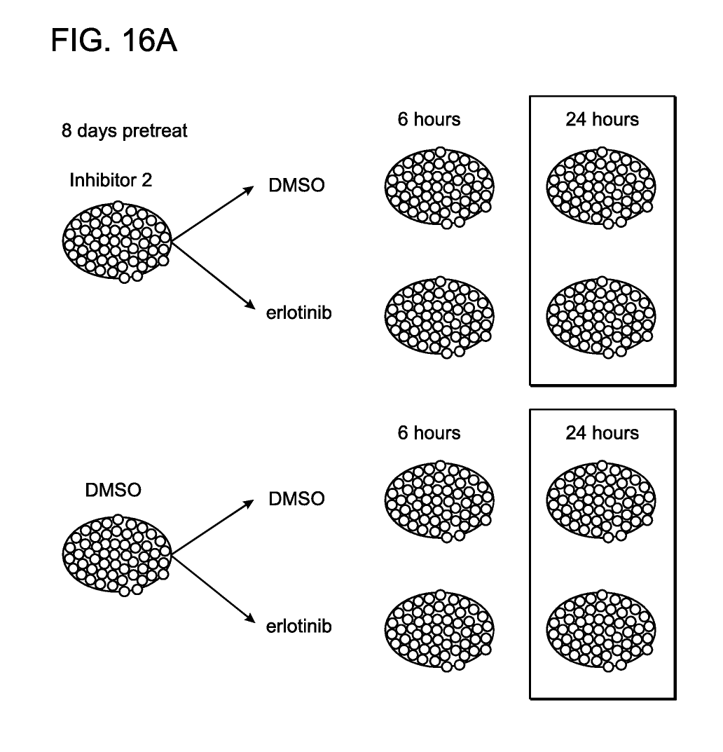

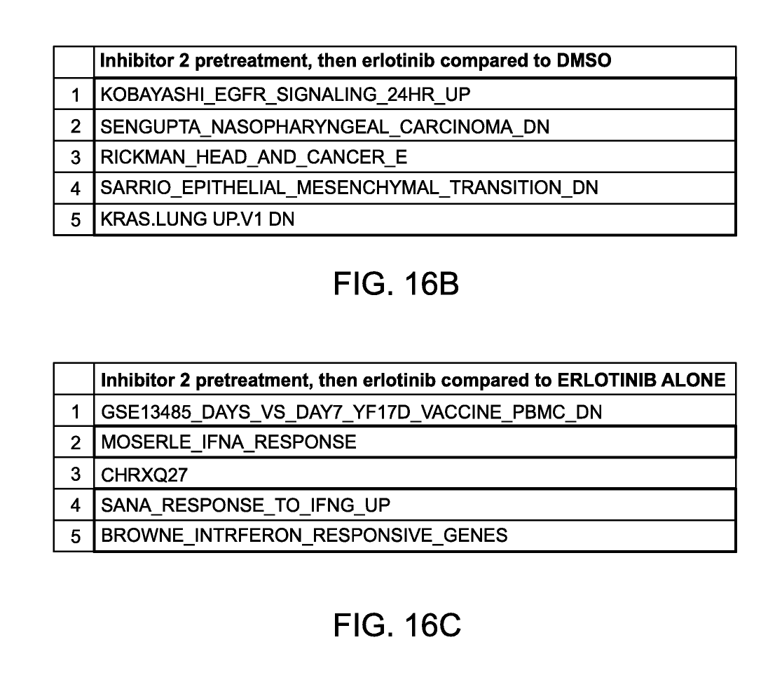

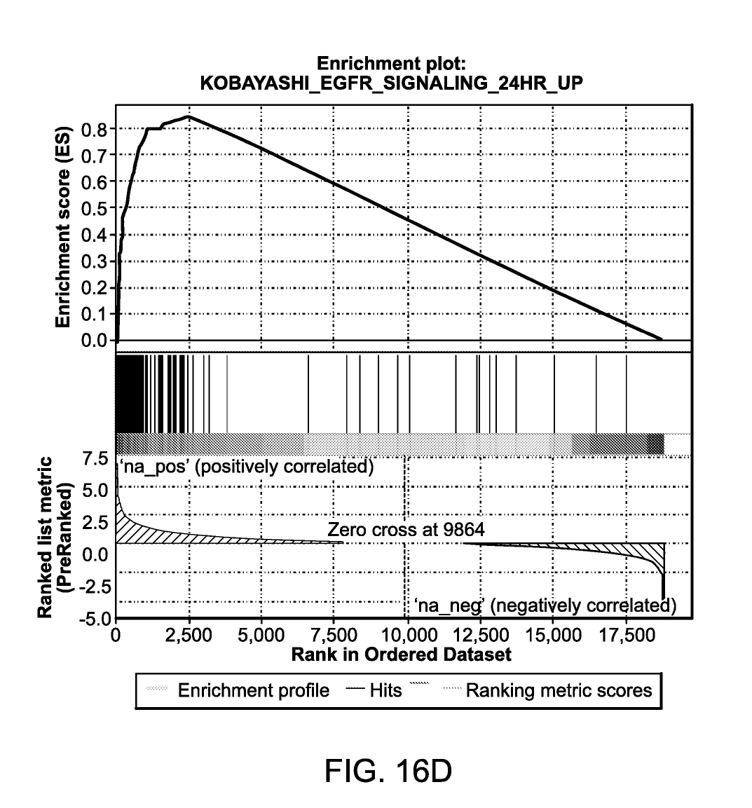

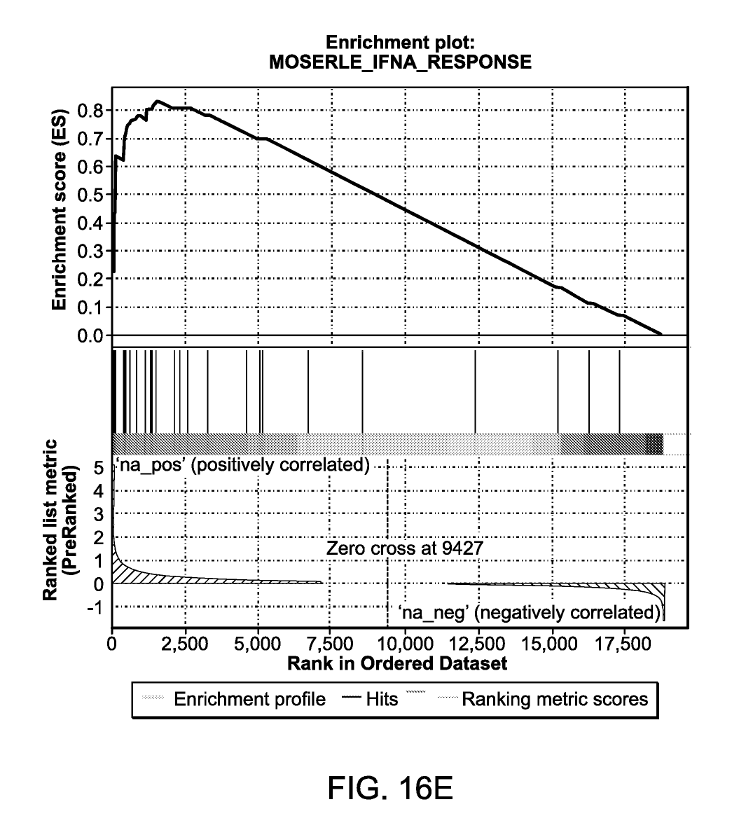

PC9 cells were pre-treated with 2.5 μM Inhbitor 2 or DMSO for 8 days, with splitting, re-seeding and compound re-fresh at day 4. See FIG. 16a. After 8 days, cells were acutely treated with 1 μM erlotinib or DMSO, and RNA samples harvested at 6 and 24 hours. RNA was extracted, and analyzed via RNA-sequencing, then gene set enrichment analysis using msigdb v4.0 (all categories). Comparing EZH2 inhibitor pre-treated/erlotinib acutely treated cells to DMSO pre-treat/acutely treated cells shows alteration of the EGFR signaling pathway remains the top altered pathway. See FIG. 16b. However, when comparing EZH2 inhibitor pre-treated/erlotnib acutely treated cells to DMSO pre-treated/erlotinib acutely treated cells reveals that 3 out of the top 5 altered pathways are interferon-related pathways. See FIG. 16c. Representative gene set enrichment analysis plot for EGFR pathway from FIG. 16b. See FIG. 16d. Representative gene set enrichment analysis plot for IFN pathway from FIG. 16c. See FIG. 16e.

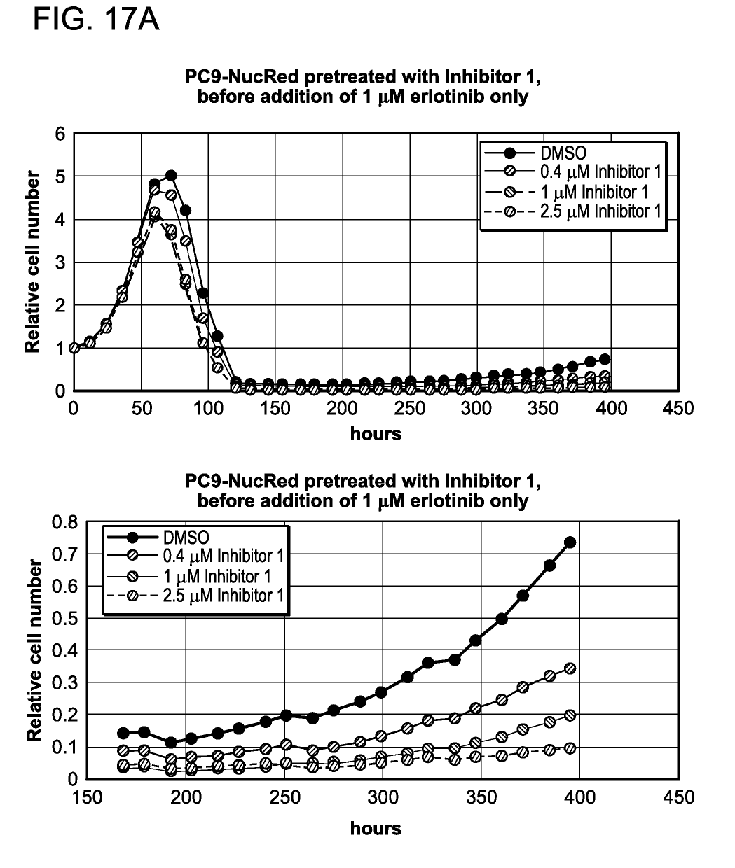

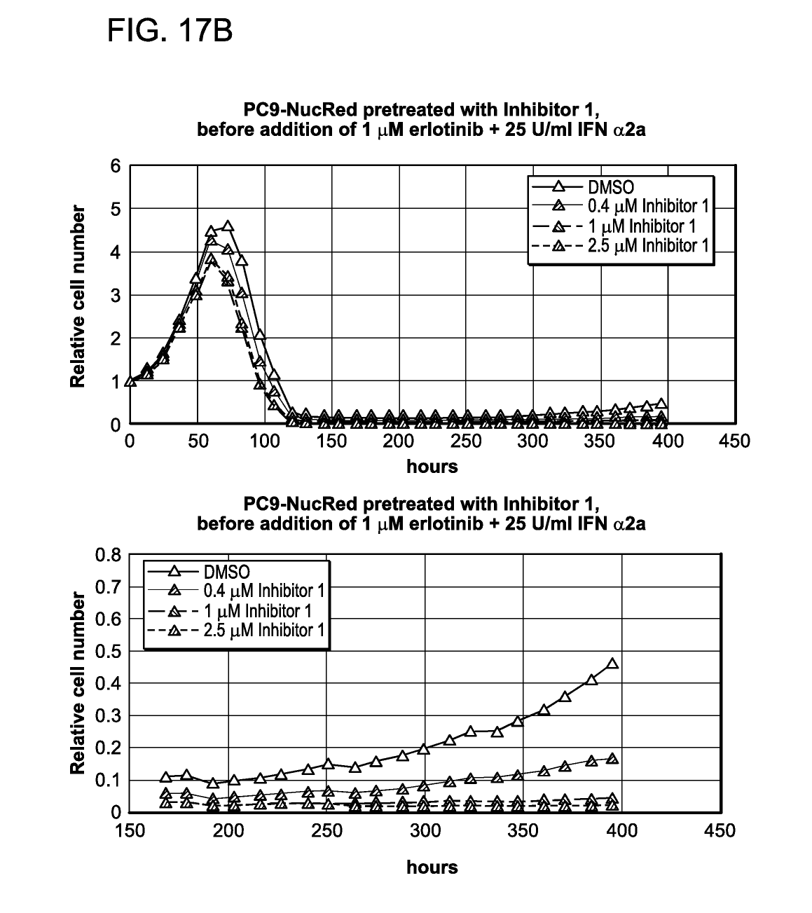

PC9 NucRed cells pre-treated with DMSO or titration of Inhibitor 1 for 3 days before re-seeding and addition of 1 μM erlotinib only or 1 μM erlotinib and 25 U/ml IFN α2a. See FIG. 17a and FIG. 17b. Media with fresh compounds was changed every 3-4 days. Phase and fluorescent images obtained every 12 hours with Incucyte imaging platform. Cell number determined by red fluorescent nuclei detection by Incucyte. Top graphs shows full time course, bottom graphs zoom to time points relevant to DTP formation and resistant cell outgrowth. Addition of 25 U/ml IFN on top of EZH2 inhibitor pre-treament lead to reduced DTP number and erlotinib-resistant cell outgrowth, compared to EZH2 inhibitor pre-treatment alone.

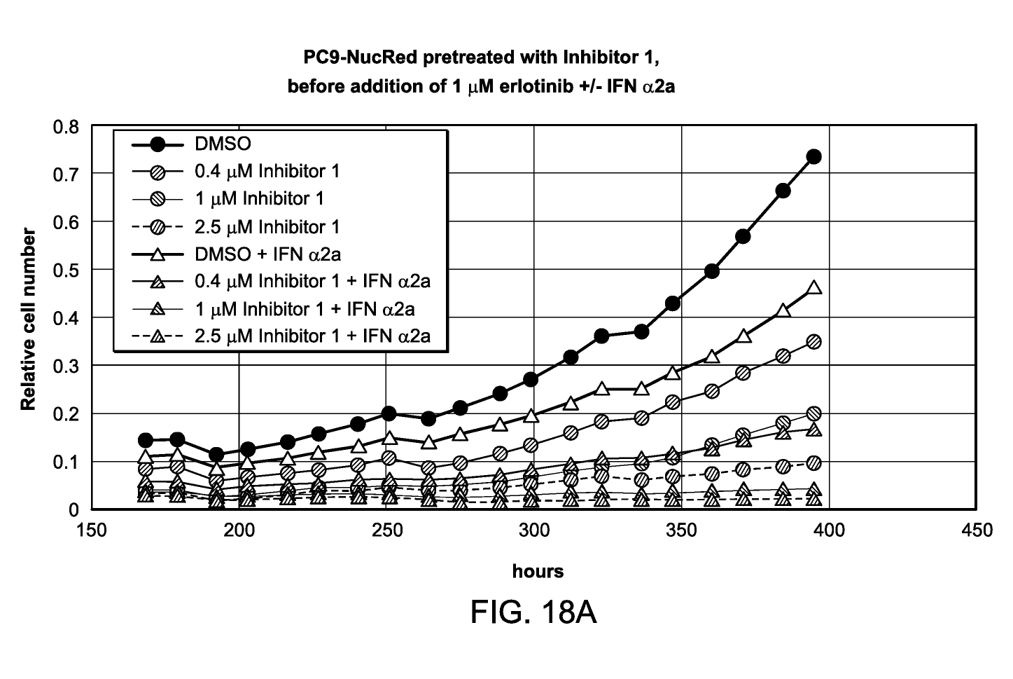

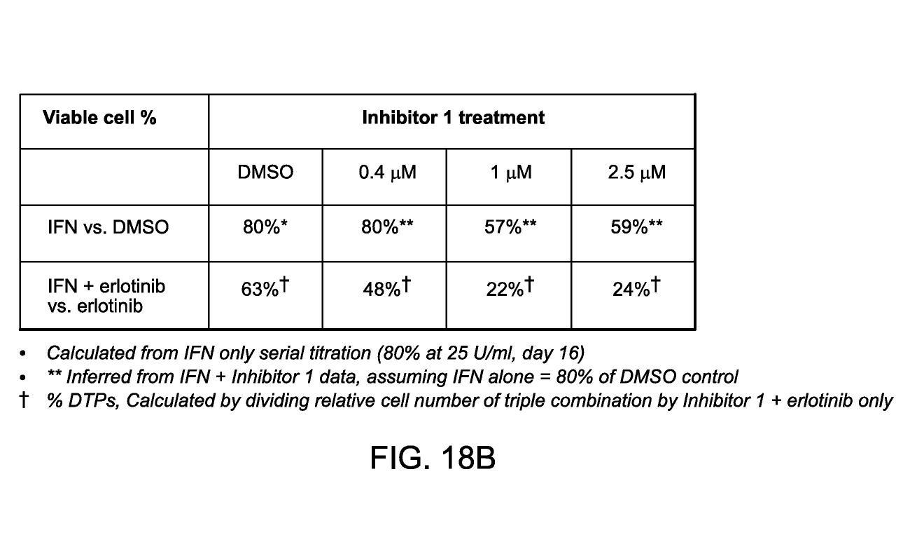

FIG. 18a represents a combination of bottom graphs from FIG. 16a and FIG. 16b, comparing PC9 NucRed erlotinib-resistant cell formation following EZH2 inhibitor pre-treatment alone (closed circles) vs. EZH2 inhibitor pretreatment plus addition of 25 U/ml IFN α2a (open triangles). At each dose level, addition of IFN leads to further reduction of DTP number and erlotinib-resistant cell outgrowth when compared to EZH2 inhibitor treated cells alone. See FIG. 18b. Table summarizing PC9 NucRed viable cell % under various conditions. Cells were pre-teated with DMSO or various concentrations of Inhibitor 1, then subjected to treatment with DMSO, 25 U/ml IFN α2a alone, or IFN and 1 μM erlotinib. Cells treated with 25 U/ml IFN α2a alone results in 80% cell viability at day 16 Pre-treatment with 0.4 μM Inhibitor 1, then 25 U/ml IFN results in 80% cell viability, whereas pre-treatment with 1 or 2.5 μM Inhibitor 1, then 25 U/ml IFN results in 57-59% cell viability. Pre-treatment with DMSO, followed by addition of 25 U/ml IFN and erlotinib results in 63% DTP number compared to erlotinib treatment alone, suggesting a synergistic effect of IFN+erlotinib compared to erlotinib only. Pre-treatment with Inhibitor 1 followed by addition of IFN and erlotinib results in further reduction of DTP number compared to Inhibitor 1 pre-treatment and erlotinib treatment, suggesting further synergy of IFN plus EZH2 inhibitor in reducing erlotinib DTP number. Results: In each case the triple combination has a greater effect than the double combination of Inhibitor 1+erlotinib. Comparing the triple combination to IFN+EZH2 inhibitor alone at each dose level indicates a greater than additive result. This indicates the addition of EZH2 inhibitor and IFN significantly reduces (if not eliminates) the erlotinib-treated DTP population in PC9 cells.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A method of treating cancer in a subject in need thereof comprising the step of administering to the subject in need thereof an effective amount of an EZH2 inhibitor and an effective amount of a type I interferon.

2. The method of claim 1, wherein the type I interferon is an alpha or beta interferon encoded by genes selected from IFNA1, IFNA2, IFNA4, IFNA5, IFNA6, IFNA7, IFNA8, IFNA10, IFNA13, IFNA14, IFNA16, IFNA17, IFNA21, IFNB1, IFNW1, IFNE, and IFNK.

3. The method of claim 1, wherein the type I interferon is interferon-alpha-2a, interferon-alpha-2b, or interferon-beta-1a.

4. The method of claim 1, wherein the type I interferon is pegylated interferon-alpha-2a, pegylated interferon-alpha-2b, or pegylated interferon-beta-1a.

5. The method of claim 1, wherein the type I interferon is peginterferon alfa-2a (Pegasys) or peginterferon alfa-2b (Peg-Intron).

6. The method of claim 1, wherein the EZH2 inhibitor is selected from EPZ-6438, EPZ005687, EPZ011989, EI1, GSK126, GSK343, and UNC1999.

7. The method of claim 1, wherein the EZH2 inhibitor is selected from 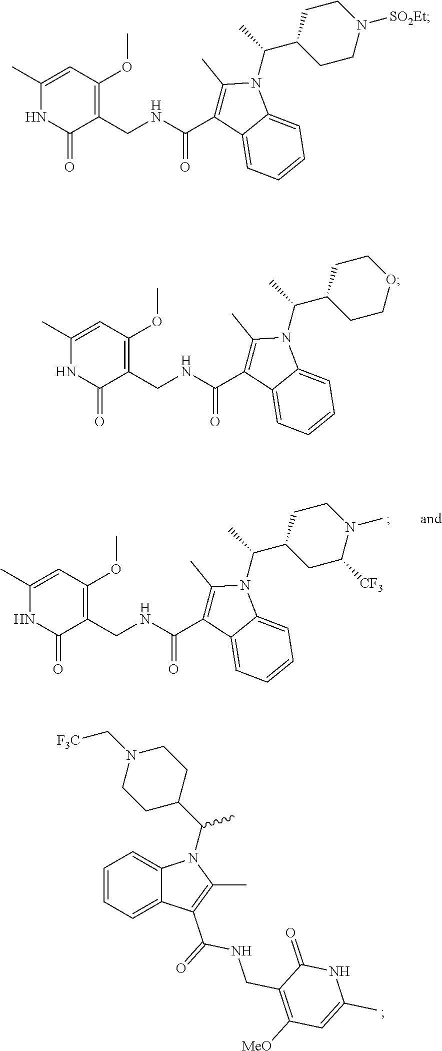
or a pharmaceutically acceptable salt thereof.

8. The method of claim 1, wherein the EZH2 inhibitor is 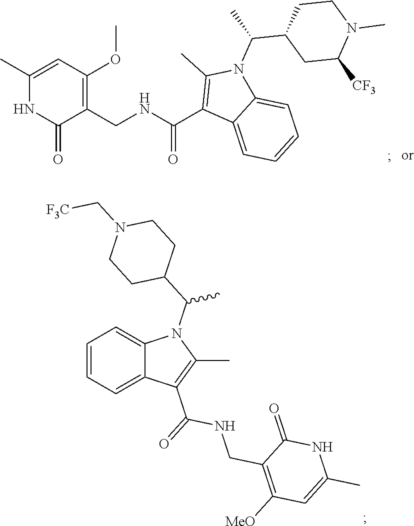
or a pharmaceutically acceptable salt thereof.

9. The method of claim 1, wherein the EZH2 inhibitor is administered concurrently with the type I interferon.

10. The method of claim 1, wherein the cancer is selected from multiple myeloma, Hodgkin's lymphoma, non-Hodgkin's lymphoma, chronic lymphocytic leukemia, adult acute myeloid leukemia (AML), acute B lymphoblastic leukemia (B-ALL), and T-lineage acute lymphoblastic leukemia (T-ALL).

11. The method of claim 1, wherein the cancer is selected from Hodgkin's lymphoma, non-Hodgkin's lymphoma, chronic lymphocytic leukemia, and multiple myeloma.

12. The method of claim 1, wherein the cancer is non-Hodgkin's lymphoma.

13. The method of claim 1, wherein the cancer is selected from melanoma, prostate cancer, breast cancer, ovarian cancer, colon cancer, bladder cancer, lung adenocarcinoma, and carcinoma of the pancreas.

14. A pharmaceutical composition comprising an effective amount of an EZH2 inhibitor and a type I interferon.

15. The pharmaceutical composition of claim 14, wherein the type I interferon is an alpha or beta interferon encoded by genes selected from IFNA1, IFNA2, IFNA4, IFNA5, IFNA6, IFNA7, IFNA8, IFNA10, IFNA13, IFNA14, IFNA16, IFNA17, IFNA21, IFNB1, IFNW1, IFNE, and IFNK.

16. The pharmaceutical composition of claim 14, wherein the type I interferon is interferon-alpha-2a, interferon-alpha-2b, or interferon-beta-1a.

17. The pharmaceutical composition of claim 14, wherein the type I interferon is pegylated interferon-alpha-2a, pegylated interferon-alpha-2b, or pegylated interferon-beta-1a.

18. The pharmaceutical composition of claim 4, wherein the type I interferon is peginterferon alfa-2a (Pegasys) or peginterferon alfa-2b (Peg-Intron).

19. The pharmaceutical composition of claim 14, wherein the EZH2 inhibitor is selected from EPZ-6438, EPZ005687, EPZ011989, EI1, GSK126, GSK343, and UNC1999.

20. The pharmaceutical composition of claim 14, wherein the EZH2 inhibitor is selected from 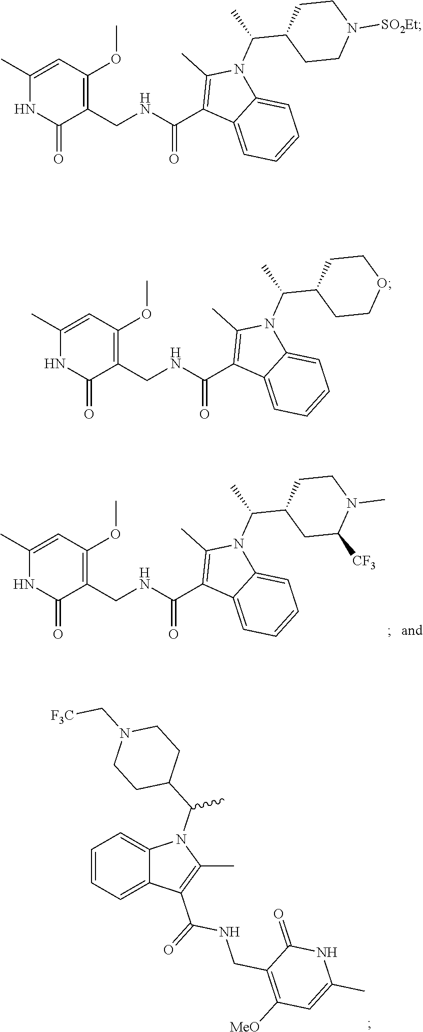
or a pharmaceutically acceptable salt thereof.

21. The pharmaceutical composition of claim 14, wherein the EZH2 inhibitor is 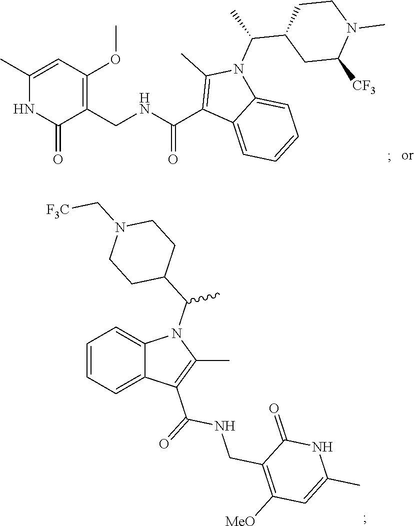
or a pharmaceutically acceptable salt thereof.
